(19) United States
(12) Patent Application Publication
(10) Pub. No.: US 2023/0016526 A1
(43) Pub. Date: Jan. 19, 2023

(54) SYSTEM AND METHODS FOR MANIPULATION OF THE ORAL MICROBIOME IN MAMMALS

(71) Applicant: ANIMAL MICROBIOME ANALYTICS, INC. (D/B/A ANIMALBIOME), Oakland, CA (US)

(72) Inventors: H. H. GANZ, Oakland, CA (US); C. X. OSBORNE, Oakland, CA (US)

(21) Appl. No.: 17/782,163

(22) PCT Filed: Dec. 3, 2020
(86) PCT No.: PCT/US2020/063158

§ 371 Date: Jun. 2, 2022

Related U.S. Application Data
(60) Provisional application No. 62/942,993, filed on Dec. 3, 2019.

(51) Int. Cl. G01N 33/569; C12Q 1/689

(52) U.S. Cl. CPC G01N 33/56955; C12Q 1/689; C12Q 2600/106

(57) ABSTRACT

This invention is directed to manipulation and modification of the oral microbiome of a mammal, in particular a companion mammal, by intervention, for example by administration of supplemental compounds, modification of a dietary component, and/or physical intervention, based on sampling and assessment of microbes. The methods can be for the purpose of potentially improving an oral health condition of the individual. Manipulations are based on analysis of at least one sample obtained from an oral tissue or oral cavity of the mammal, and optionally other mammals, each sample including or indicative of microbes of an oral microbiome.

### CROSS-REFERENCE TO RELATED APPLICATION

This application claims priority to and the benefit of U.S. provisional patent application No. 62/942,993, filed on Dec. 3, 2019, which is incorporated by reference herein in its entirety, and expressly including any drawings, for all purposes.

### BACKGROUND

### Field of the Invention

This invention is directed to manipulation and modification of the oral microbiome of a mammal, in particular a companion mammal, by intervention, for example by administration of supplemental compounds, modification of a dietary component, and/or physical intervention, based on sampling and assessment of microbes.

### Description of the State of the Art

In mammals, the oral cavity is a moist environment characterized by constant temperature (34 to 36° C.) and a neutral pH that connects to the respiratory and digestive systems. Food is taken into the oral cavity and may be ground or torn into pieces before being swallowed. In most mammals, the oral cavity includes hard surfaces (e.g. teeth, above and below the gumline) as well as soft mucosal surfaces (e.g. the cheek, tongue, gingiva, palate, and floor of the mouth). These surfaces can be colonized by microbes, such as those found in food or water taken into the oral cavity.

The oral cavity of mammals is known to host one or more communities or collections of microbes, including bacteria, archaea, fungi, the various single-celled eukaryotes generally referred to as protists, and viruses. A community or collection of microbes existing in the oral cavity is referred to as the oral microbiome. An oral microbiome of a mammal may vary over time and oral microbiomes vary among individuals. An oral microbiome can be characterized by various features, such as the identity and/or relative abundance of its microbial taxa or their functions or metabolic byproducts.

Bacteria found in an oral microbiome of a mammal can have beneficial, commensal, or pathogenic effects on an individual's health, in particular, its oral health. For example, bacteria living in oral biofilm communities may help provide protection from invasion by potential pathogens. Also for example, an increase in gram-negative anaerobic bacteria may contribute to gum disease. The quality of an individual's oral health can affect the individual's overall health and social well-being, as poor oral health can cause bad breath and limit the individual's ability to bite, chew, swallow and/or vocalize.

### SUMMARY OF THE INVENTION

Embodiments of the invention encompass methods for manipulating or modifying an oral microbiome of an individual mammal, in particular a companion mammal, including for the purpose of potentially improving an oral health condition of the individual, by identifying an intervention or a plan including interventions based on analysis of at least one sample obtained from an oral tissue or oral cavity of the mammal, and optionally other mammals, each sample including or indicative of microbes of an oral microbiome.

A sample including or indicative of microbes (for example, a “test” sample) is analyzed to obtain data (for example, “test” data) for a plurality of taxa of the microbes of an oral microbiome of the individual. In a preferred embodiment, the analyzing comprises analyzing genetic material. The sample or a portion thereof, for example, an extract or fraction of one or more nucleic acids, can be prepared for analysis, for example, nucleic acids of the test sample can be amplified and/or sampled. In a further preferred embodiment, the analyzing comprises identifying the plurality of taxa of microbes by sequence analysis of the genetic material. The sequences can be used to identify or measure a particular microbial taxon or group of taxa. The data obtained from the sample represent or indicate or otherwise reflect a plurality of taxa of microbes of an oral microbiome of the individual.

In some embodiments, the genetic material includes deoxyribonucleic acid (DNA) from microbes of the oral microbiome of the companion mammal. In some embodiments, identifying the plurality of taxa of microbes is accomplished by shotgun sequencing of extracted DNA and/or by targeted sequencing of microbial diversity. In some embodiments, the genetic material includes ribonucleic acid (RNA) from microbes of the oral microbiome of the companion mammal. In some embodiments, the analyzing comprises analyzing proteins in the test sample and each of the one or more reference samples. In some embodiments, the analyzing further comprises characterization of metabolites, for example, volatile metabolites, for example, using chemical analysis. In some embodiments, the analyzing further comprises characterizing the composition or metabolic function of the oral microbiome of a biofilm of the companion mammal.

A sample including or indicative of microbes can be preserved, for example, one or more nucleic acids from the sample can be preserved. In some embodiments, preserving includes drying the sample, placing the sample in a buffer and/or combining the test sample with a preservative, for example, ethanol, nucleic acid preservation (NAP) buffer, CD1, or any of a variety of commercially available buffers, in particular, those for preserving nucleic acids.

In some embodiments, a measure (for example, a “test” measure) is determined for each of one or more microbiome features based on data obtained from the sample (for example, “test” data). In some embodiments, a measure is determined for each of one or more microbiome features based on data obtained from the sample and other data, for example, a reference measure. A measure for a microbiome feature can be a value, a set of values, a range of values, a normalized value, a parameter or a set of parameters for a statistic, metric, or model, or any combination thereof. A measure for a microbiome feature can be numeric and/or non-numeric, and can be quantitative and/or qualitative. A measure for a microbiome feature is preferably based on data for two or more taxa of microbes, or includes values for two or more taxa of microbes.

A measure for a microbiome feature can indicate or reflect one or more aspects of the composition of microbes in the oral microbiome and/or changes in the composition of microbes in the oral microbiome. A measure for a microbiome feature can include one or more indicators of alpha diversity or any indicator of taxonomic diversity, richness, and/or evenness, for example, number of taxa, and/or changes in such indicators. A measure for a microbiome feature can include an indication of similarity and/or relatedness of two or more of the plurality of taxa of the microbes of the oral microbiome, and/or change in such indicators. A measure for a microbiome feature can include an indication of presence or absence of a taxon, presence of each of a plurality of taxa, and/or presence or absence of each of a plurality of predefined taxa, and/or change in such indicators. A measure for a microbiome feature can include one or more indicators of amount or relative abundance of one or more of the plurality of taxa, and/or change in such indicators.

A measure for a microbiome feature can indicate or reflect one or more aspects of the function of microbes in the oral microbiome, for example based on microbial metabolic products, or changes in such function, for example, based on changes in microbial metabolic products, for example, as shown in FIGS. 3 and 4. A measure for a microbiome feature can include one or more indicators of activity for one or more metabolic categories of microbes in the oral microbiome, and/or change in such indicators. A measure for a microbiome feature can include one or more indicators of anaerobic microbes, aerobic microbes, periodontal disease-associated microbes, pathogen/pathobiont microbes, and/or oral health-associated microbes, and/or change in such indicators. A measure for a microbiome feature can include one or more indicators of anaerobic bacteria, aerobic bacteria, periodontal disease-associated bacteria, pathogen/pathobiont bacteria, and/or oral health-associated bacteria, and/or change in such indicators.

A measure for a microbiome feature can indicate or reflect one or more aspects of the composition of microbes for a category of microbes in the oral microbiome and/or changes in the composition of microbes for a category in the oral microbiome, for example as shown in FIGS. 3 and 4. For example, a measure for a microbiome feature can include an indication of number of taxa and/or presence or absence of one or more taxa of microbes for each of a plurality of categories, and/or changes in such indicators. A measure for a microbiome feature can include one or more indicators of amount or relative abundance of microbes, for example by taxa, for one or more categories, and/or change in such indicators. A measure for a microbiome feature can include or reflect an indicator of one or more indicators of alpha diversity or any indicator of taxonomic diversity, richness, and/or evenness, and/or changes in such indicators, for each of one or more categories of microbes. A measure for a microbiome feature can include an indication of similarity and/or relatedness of two or more of the plurality of taxa for each of one of more categories of the microbes of the oral microbiome, and/or change in such indicators.

For example, in some embodiments, a microbiome feature indicates the number of taxa and/or an array and/or a change in an array of aerobic microbes, anaerobic microbes, periodontal disease-associated, pathogen/pathobiont microbes, and/or oral health-associated microbes present and/or absent in a sample. In some embodiments, a microbiome feature indicates a relative abundance and/or a change in relative abundance of aerobic microbes, anaerobic microbes, periodontal disease-associated, pathogen/pathobiont microbes, and/or oral health-associated microbes. In some embodiments, a microbiome feature indicates taxonomic diversity, richness, and/or evenness for one of more of aerobic microbes, anaerobic microbes, periodontal disease-associated, pathogen/pathobiont microbes, and/or oral health-associated microbes. In some embodiments, a microbiome feature includes an indication of similarity and/or relatedness of taxa, and/or change in such indicators, for aerobic microbes, anaerobic microbes, periodontal disease-associated, pathogen/pathobiont microbes, and/or oral health-associated microbes.

In some embodiments, the microbiome feature includes only bacteria. In some embodiments, a microbiome feature indicates the number of taxa and/or an array and/or a change in an array of aerobic bacteria, anaerobic bacteria, periodontal disease-associated bacteria, pathogen/pathobiont bacteria, and/or oral health-associated bacteria present and/or absent in a sample. In some embodiments, a microbiome feature indicates a relative abundance and/or a change in relative abundance of aerobic bacteria, anaerobic bacteria, periodontal disease-associated bacteria, pathogen/pathobiont bacteria, and/or oral health-associated bacteria. In some embodiments, a microbiome feature indicates taxonomic diversity, richness, and/or evenness for one of more of aerobic bacteria, anaerobic bacteria, periodontal disease-associated bacteria, pathogen/pathobiont bacteria, and/or oral health-associated bacteria. In some embodiments, a microbiome feature includes an indication of similarity and/or relatedness of taxa, and/or change in such indicators, for aerobic bacteria, anaerobic bacteria, periodontal disease-associated bacteria, pathogen/pathobiont bacteria, and/or oral health-associated bacteria.

A measure for a microbiome feature can be a combination of measures for a particular category, for example, a listing of number of taxa, diversity, and relative abundance for each of one or more categories of microbes. A measure for a microbiome feature can be a combination of measures from two or more categories, for example, a combined measure of diversity for all favorable categories of microbes, for example, oral-health associated microbes and aerobic microbes. Also for example, a measure for a microbiome feature can be a ratio of measures, or combined measures, for each of two or more categories, for example, a ratio of taxonomic diversity, richness, or evenness of periodontal disease-associated microbes to taxonomic diversity, richness, or evenness of oral health-associated microbes.

In some embodiments, one or more reference measures are provided for each of one or more microbiome features. A reference measure can be based on analysis of one or more reference samples from oral tissues or oral cavities of one or more reference mammals, the one or more reference samples including microbes of one or more reference oral microbiomes, the analysis of the one or more reference samples providing reference data for a plurality of taxa of microbes, the at least one reference measure based on the reference data. A reference measure can be based on analysis of one or more reference samples from the oral tissue or cavity of an individual being tested and/or from oral tissues or oral cavities of one or more other mammals, for example, from two or more reference mammals. The mammals, including the individual being tested and/or one or more reference mammals, can be characterized according to their health, for example, by oral health state or by periodontal disease state.

In some embodiments, a threshold value or range of values is identified based on one or more reference measures. For example, for a microbiome feature defined as the ratio of richness of periodontal disease-associated bacteria to richness of oral health-associated bacteria, a threshold value may be 1.2. Measures of this microbiome feature that are higher than, for example, about 1.2, can indicate an unfavorable microbiome and measures that are lower than, for example, about 1.2, can indicate a favorable microbiome.

In some embodiments, the sample obtained from the oral tissue or oral cavity of the individual mammal is obtained after a preliminary intervention. In some embodiments, the one or more reference samples, or at least one of the reference samples, is obtained prior to a preliminary intervention. In some embodiments, at least one of the reference samples is obtained after a preliminary intervention.

One or more interventions can be identified based on assessment of data obtained from one or more samples obtained from the oral tissues or cavities of one or more mammals. In some embodiments, an intervention is identified based on at least one test measure and at least one reference measure for one or more microbiome features.

Interventions can be identified with the goal or intent of modifying the oral microbiome of the individual. In some embodiments, the identified intervention is implemented. In some embodiments, the oral microbiome of the companion mammal is modified by implementation of the identified intervention. Modification of the oral microbiome can include improving at least one of the one or more microbiome feature of the oral microbiome of the companion mammal. An identified intervention can be selected to modify the oral microbiome of the companion mammal, for example, by improving the balance of microbes of the oral microbiome, by increasing favorable microbes, and/or by decreasing unfavorable microbes.

In some embodiments, an identified intervention is the same as or different from a preliminary or prior intervention, or in addition to a preliminary or prior intervention. In some embodiments, an intervention is identified where a prior intervention is determined to be unsuccessful. In some embodiments, a plan for the companion mammal's oral health is provided. The plan can include one or more identified interventions.

Identifying an intervention can include assessing the success of a prior or preliminary intervention, for example, by assessing whether there is an improvement in a measure of microbes in at least one category. In some embodiments, the category is selected from aerobic microbes, anaerobic microbes, periodontal-associated microbes, pathogen/pathobiont microbes, and oral-health associated microbes. In some embodiments, the category is selected from aerobic bacteria, anaerobic bacteria, periodontal-associated bacteria, pathogen/pathobiont bacteria, and oral-health associated bacteria. In some embodiments, success of a prior or preliminary intervention is indicated by one or more of an increase in aerobic microbes, an increase in oral-health associated microbes, a decrease in anaerobic microbes, a decrease in periodontal disease-associated microbes, and/or a decrease in pathogen/pathobiont microbes. In some embodiments, success of a prior or preliminary intervention is indicated by one or more of an increase in aerobic bacteria, an increase in oral-health associated bacteria, a decrease in anaerobic bacteria, a decrease in periodontal disease-associated bacteria, and/or a decrease in pathogen/pathobiont bacteria. In some embodiments, assessing the success of the preliminary intervention includes determining one or more of an increase in taxonomic diversity, taxonomic richness, and taxonomic evenness for the oral microbiome or for one or more categories of microbes in the oral microbiome. In some embodiments, assessing the success of the preliminary intervention includes consideration of more or more of the companion mammal's diet, general health, exposure to microbes, dental care history, and oral hygiene. Oral hygiene can include one or more of health of teeth, health of gums, health of tongue, low or no plaque and tartar, periodontal health, and fresh breath.

The identified intervention and/or the preliminary intervention can include administration of a probiotic, prebiotic fiber, prebiotic supplement, bacteriophage, bacteriophage-associated protein, bacteriophage lysins, antimicrobial, antibiotic, antimicrobial peptide, nanoparticles with biocidal properties, sodium bicarbonate, chelating agents, glycoproteins, phototherapy, and any combination thereof. The identified intervention and/or the preliminary intervention can include modification of a dietary component, for example, removal, addition, or limitation of a dietary component. The identified intervention and/or the preliminary intervention can include tooth brushing, tooth flossing, mechanical removal or reduction in biofilm, dental cleaning, tooth extraction, root canal, dental examination, administration of an antibiotic, and any combinations thereof. In some embodiments, material for executing an intervention are provided. Such material can include a compound, medicine, supplement, prescription, literature, device, tool, implement, and/or kit. In some embodiments, the method includes providing material for obtaining a sample, for example, a sampling kit.

In some embodiments, the companion mammal is a dog. In some embodiments, the companion mammal is a cat. In some embodiments, the companion mammal is an equine. In some embodiments, the companion mammal is a ferret. In some embodiments, the companion mammal is a rabbit. In some embodiments, the companion mammal and the one or more reference companion mammals are of a same species. In some embodiments, the companion mammal and at least one of the one or more reference companion mammals are of a same breed. In some embodiments, the companion mammal and at least one of the one or more reference companion mammals are of different breeds.

Embodiments of the invention further encompass methods for manipulating an oral microbiome in an individual mammal, and methods for improving an oral health condition of an individual mammal, by obtaining a first sample from an oral tissue or oral cavity of the individual that includes microbes of the oral microbiome at a first time, obtaining a second sample from the tissue or cavity of the individual that includes microbes of the oral microbiome at a second time, analyzing the first and second samples to determine one or more microbiome features, and based on the one or more microbiome features, identifying an intervention to alter the oral microbiome and/or to improve the oral health of the individual.

Such embodiments can further comprise obtaining a third sample from the tissue or cavity of the individual that includes microbes of the oral microbiome at a third time, analyzing the third sample to identify any changes to the one or more microbiome features, and using the third sample to identify a new intervention to alter the oral microbiome of the individual. The new intervention can be the same or different than the intervention identified based on the microbiome features determined from analysis of the first and second samples.

Embodiments of the invention encompass methods for improving an oral health condition of an individual mammal, and methods for manipulating an oral microbiome in an individual mammal, by obtaining a sample from an oral tissue or oral cavity of the individual that includes microbes of the oral microbiome, analyzing the sample to determine one or more microbiome features, obtaining reference values for the one or more microbiome features that are based on analysis of samples from oral tissues or oral cavities of a plurality of individuals similar to the individual mammal, and, based on the one or more microbiome features determined from analysis of the sample and the reference values for the one or more microbiome features, identifying an intervention to improve the oral health condition and/or to alter the oral microbiome of the individual.

Such embodiments can further comprise obtaining a second sample from the tissue or cavity of the individual that includes microbes of the oral microbiome at a second time, analyzing the second sample to determine one or more new values of the one or more microbiome features, and, based on the one or more new values of the one or more microbiome features, identifying a new intervention to improve the oral health condition and/or to alter the oral microbiome of the individual.

In some embodiments, the second sample is obtained from about 1 week, 2 weeks, 3 weeks, 4 weeks, 5 weeks, 6 weeks, 7 weeks, 8 weeks, 3 months, 6 months, 9 months, or 12 months after the first sample is obtained. In some embodiments, the third sample is obtained from about 1 week, 2 weeks, 3 weeks, 4 weeks, 5 weeks, 6 weeks, 7 weeks, 8 weeks, 3 months, 6 months, 9 months, or 12 months after the second sample is obtained.

In some embodiments, the reference values are based on analysis of samples obtained from a plurality of individuals that do not have the oral health condition. In some embodiments, the reference values are based on analysis of samples obtained from plurality of individuals that have received the intervention. In some embodiments, analyzing the sample from the individual includes use of analysis methods similar to those used in analysis of samples from oral tissues or oral cavities of the plurality of individuals.

In some embodiments, identifying an intervention and/or a new intervention includes consideration of the individual's condition, for example, between the first and second time, or between the second and third time. The individual's condition can include at least one of: diet, general health, exposure to microbes, dental care history, and oral hygiene. In some embodiments, the intervention is administration of a dietary component, or a dental cleaning, or a change in oral hygiene practice. In some embodiments, the dietary component or new dietary component comprises at least one of: a prebiotic fiber, a plant-derived fiber, and an animal-derived fiber. In some embodiments, the dental care history is time since last dental cleaning. In some embodiments, oral hygiene is frequency of tooth brushing or flossing.

For example, in one embodiment, the individual's condition includes diet, the intervention is administration of a dietary component, and the method further comprises providing the dietary component in a form suitable for consumption by the individual. Also for example, in one embodiment, the individual's condition is time since last dental cleaning and the intervention is more frequent tooth brushing or flossing.

Embodiments of the invention can include altering the oral microbiome in the individual by making the intervention with the individual. Embodiments of the inventions can include improving an oral health condition of the individual. In some embodiments, the oral health condition is periodontal disease. In some embodiments, interventions to improve the oral health condition of the individual include interventions to generate a beneficial biofilm in the oral cavity of the individual.

### BRIEF DESCRIPTION OF THE DRAWINGS

FIG. 1 is a flow chart for an exemplary embodiment of the invention.

FIG. 2 is a flow chart for another exemplary embodiment of the invention.

FIG. 3 is a diagram showing exemplary categories of microbes that could be used in a microbiome feature, with exemplary genera of microbes listed for each category.

FIG. 4 is a diagram providing examples of the types of indicators that could be used in a microbiome feature, including indicators for phylogenetic and phenotypic data.

FIG. 5 provides an example of an assessment of microbiome features for a dog using sequential sampling.

FIG. 6 provides another example of an assessment of microbiome features for a dog using sequential sampling.

### DETAILED DESCRIPTION OF THE INVENTION

This patent describes systems and methods for manipulating or modifying the oral microbiome of an individual mammal, in particular, a companion mammal, by identifying an intervention, or a plan including interventions, based on analysis of at least one sample obtained from an oral tissue or oral cavity of the mammal, and optionally other mammals, for example, to promote oral health.

As used herein, the term “microbiome” refers to a collection or community of microbes of a particular region of an individual's body. The microbes of a microbiome include at least bacteria, and may also include, but are not limited to including, archaea, viruses, fungi, protists, or a combination thereof.

The oral microbiome includes any community or collection of microbes existing in the oral cavity and/or oral tissues of an individual, including without limitation the mouth, throat, gums, teeth, lips, cheeks, and tongue regions. There may be multiple oral microbiomes in the oral cavity of an individual, e.g. the oral microbiome of the gums or the oral microbiome of the throat. As used herein, the term “oral microbiome” includes any localized oral microbiome, any combination of localized oral microbiomes, and the entirety of microbiomes occurring in the oral cavity and oral tissues of an individual.

As used herein, “mammal” or “mammals” refers to any member of the Class Mammalia, including without limitation any individual, breed, population, sub-phylum or other subgroup of the Class Mammalia.

As used herein, “non-human mammal” refers to any member of the Class Mammalia excluding the taxon Homo sapiens, as a whole, as well as any individual, race, population, or other subgroup of the taxon Homo sapiens.

As used herein, “domestic” mammal refers to any non-human mammal that is selectively bred or managed by humans and/or that lives with humans, and includes without limitation companion mammals, livestock mammals, and captive or managed mammalian wildlife. Examples of livestock mammals include, but are not limited to including, cows, horses, donkeys, mules, pigs, sheep, and goats. Examples of captive or managed mammalian wildlife, include, but are not limited to including, cheetahs, wolves, lions, leopards, and elephants.

As used herein, “companion” mammal refers to any mammal that is not kept for commercial purposes and/or that serves, or is of a type that commonly serves, as a companion to or as a pet of humans. Examples of companion mammals include, but are not limited to, cats, dogs, ferrets, rabbits, equine (horses). A companion mammal can be captive or managed mammalian wildlife.

The oral microbiome is known to vary among individuals. For example, the oral microbiome varies among individual domestic dogs. The oral microbiome is also known to change over time. For example, the oral microbiome of particular individual domestic dogs are known to change over time.

The features of the oral microbiome may depend on a variety of conditions, including for example the individual's diet, general health, exposure to microbes, dental care history, and oral hygiene, and changes in these conditions. Features of the individual's diet that may influence the oral microbiome include, without limitation, the amount, proportion and/or type of fiber, refined carbohydrates, sweeteners, acidic foods, and supplements. Fibers can include bone, fur and feathers. Supplements can include prebiotics, postbiotics, antimicrobial compounds extracted from plants (such as green tea leaves, tea tree oil, eucalyptol), freshwater, and marine algae, including terpenoids, and Vitamin D supplementation. An individual's dental care history includes, without limitation, the time and frequency of dental exams and dental cleanings by a professional. Oral hygiene includes the practice of brushing of the teeth, e.g. with a toothbrush and toothpaste, and dental cleaning between the teeth, e.g. with dental floss and/or dental chews. The quality of oral hygiene depends at least in part on the frequency and regularity of such brushing and dental cleaning.

Mammals have a number of physiological adaptations that can help protect the oral environment. Gingival crevicular fluid is an inflammatory exudate found associated with the gingival crevice that contains antibodies directed against dental plaque bacteria. Saliva is 99% water but also contains hormones, proteins, glycoproteins, vitamins, urea, and ions (calcium, phosphate, magnesium, and fluoride) that help in the remineralization of tooth enamel. The rate of secretion of saliva also affects pH and the concentration of ions such as sodium, bicarbonate, potassium, calcium, phosphate, chloride, as well as urea and proteins in the oral cavity, which may foster or inhibit growth of certain microbes (Eley and Cox 2003; Khurshid et al. 2017). Changes in the diet or health status may influence how well these adaptations function.

Periodontal disease can arise from complex interactions between bacteria living in biofilms in the host gingival crevices and host immune responses. Iron is essential for bacterial growth and colonization in general, and iron and heme utilization are well known to contribute to growth and virulence in Porphyromonas gingivalis, a bacterium associated with the initiation and progression of chronic periodontal disease (Olczak et al. 2005). The microbes in the oral microbiome form biofilms on an mammal's teeth in a multi-step process (reviewed in Kačírová et al. 2019). In domestic dogs, the first bacteria to colonize include members of Neisseria, Corynebacterium, Streptococcus, and Stenotrophomonas. The most common species of the genus Neisseria to colonize the teeth are N. zoodegmatis, N. animaloris and N. weaveri. Next to colonize are members of the Actinomyces, Porphyromonas, Moraxella, Leucobacter, and the families Peptostreptococcaceae and Pasteurellaceae. Bacteria that are last to colonize include Actinomyces canis and Porphyromonas gingivicanis, which are found in biofilms associated with periodontal disease in domestic dogs.

Oral health is generally understood or characterized by the incidence or occurrence of oral health conditions, including but not limited to the incidence or occurrence of chronic mouth or facial pain, oral or throat cancer, oral infection or sores, periodontal (gum) disease, tooth decay, tooth loss, and other types of oral disease or disorder. Poor oral health can be characterized by the presence of one or more oral health conditions. The quality of oral health generally declines with the severity of an oral health condition and/or the number of oral health conditions. Good oral health can be characterized by the absence or near absence of oral health conditions and/or the lack of severity of any oral health conditions.

Features of the oral microbiome can indicate the state of oral health of an individual. Such features can serve as indicators for the oral health condition and/or the quality of oral health. Such features can indicate increased or decreased severity of an oral health condition. Such features can indicate improvement or decline in the quality of oral health.

For example, an oral microbiome feature can reflect the occurrence and/or severity of an oral health condition, such as periodontal disease. Microbial taxa that have been associated with advanced periodontal disease in cats and dogs include Porphyromonas gulae, Treponema 2 spp. (denticola, lecithinolyticum, maltophilum, medium, parvum, socranskii subsp. socranskii, spp. COT: 087, 170, 199, 201, 297, 233, 246, 247, 249, 325, 350, 352, 355, 356, 357, 358, 359, 397, FOT: 123, 144), Actinomyces spp. (canis, coleocanis, graevenitzii F0530, hordeovulneris, israelii, sp, COT 374, COT 404), Peptostreptococcus spp. and other members of the Peptostreptococcaceae (Davis et al. 2013). Porphyromonas gulae is closely related to P. gingivalis but is sufficiently different to be considered a separate species (Fournier et al. 2001, O'Flynn et al. 2015). Other periodontal disease associated bacteria include Parvimonas micra and Fusobacterium nucleatum.

Also for example, an oral microbiome feature can reflect the absence and/or lower severity of an oral health condition, or good oral health. In a study of subgingival plaque collected from dogs whose oral health ranged from healthy gingiva to mild periodontitis, Porphyromonas (particularly Porphyromonas cangingivalis), Moraxella spp. (including bovoculi), and Bergeyella were found to be associate with good oral health (Davis et al. 2013). In dogs, P. cangingivalis is the most abundant bacterium found in the oral cavity, where it is associated with healthy plaque (Davis et al. 2013). Other bacteria found in the oral cavity of healthy cats and dogs include Corynebacterium spp. (durum, mustelae, COT 422, FOT 322), Leucobacter spp. (sp COT 429), Capnocytophaga (canimorsus, cynodegmi, gingivalis, granulosa, ochracea, spp.), Streptococcus spp. (anginosus subsp, anginosus, massiliensis 4401825, minor, ratans, ovis DSM 16829, salivarius subsp. thermophilus, spp (COT: 279, 297, FOT: 345), Fusibacter spp., Fretibacterium spp., Conchiformibius spp. (kuhniae, sp. COT 289, steedae), Neisseria spp. (canis, shayeganii, zoodegmatis, animaloris, weaveri), Frederiksenia spp. (canicola, uncultured bacterium), and Pasteurella spp. (dagmatis, multocida).

An oral microbiome feature may reflect factors that contribute to the occurrence of one or more oral health conditions and/or to increased severity of one or more oral health conditions in an individual. Such features are referred to here as undesirable features of the oral microbiome, including halitosis, chronic inflammation of the gums, tooth reabsorption, and tooth loss. An oral microbiome feature may reflect factors that reduce the risk of occurrence of one or more oral health conditions in an individual. Such features are referred to here as desirable features of the oral microbiome. For example, a healthy oral biofilm provides protection or colonization resistance to pathobionts, such as Escherichia coli.

The oral microbiome of a mammal can be modified or adjusted. Reduction of undesirable features of the oral microbiome and/or creation or enhancement of desirable features of the oral microbiome may improve oral health, including oral health conditions. Actions that affect a mammal's living experience or living conditions and which may have beneficial effects on the oral microbiome of at least some mammals, in some circumstances, whether such effects are confirmed or not, are referred to here as interventions. Interventions include any change in the mammal's living experience or living conditions for which there is reason to believe that a beneficial effect on the oral microbiome could occur.

For example, an oral microbiome may be beneficially adjusted by providing a diet that includes specific prebiotic fibers and/or reduces the availability of sugars. In addition or alternatively, chelating agents can be used to bind sugars and/or heme molecules to restrict pathogen growth.

Also for example, an oral microbiome may be beneficially adjusted by regular tooth brushing or having a veterinarian, dentist or hygienist perform dental cleaning or teeth extractions. Dental plaque, tartar (calculus), and biofilms can be disrupted mechanically by tooth brushing and/or flossing and/or scraping and/or polishing and/or sonication, as well as with enzymes and detergents, such as those used in toothpastes.

Also for example, an oral microbiome may be beneficially adjusted by introducing probiotic strains of bacteria and fungi to compete and potentially exclude disease-associated organisms, including the production of compounds such as N-acetyl-L-cysteine with antibiofilm properties. Bacteriophages and associated proteins can also be used to target pathogens. Phototherapy can be used to target and restrict growth of pigmented anaerobes such as Porphyromonas spp. and Prevotella intermedia. Antimicrobials can also be used to adjust the oral microbiome and these include antibiotics, antimicrobial peptides, bacteriophage lysins, and nanoparticles with biocidal properties. Sodium bicarbonate and other chemicals can be used to shift the pH towards more alkaline values, which will affect microbiome composition as well as reduce erosion of tooth enamel.

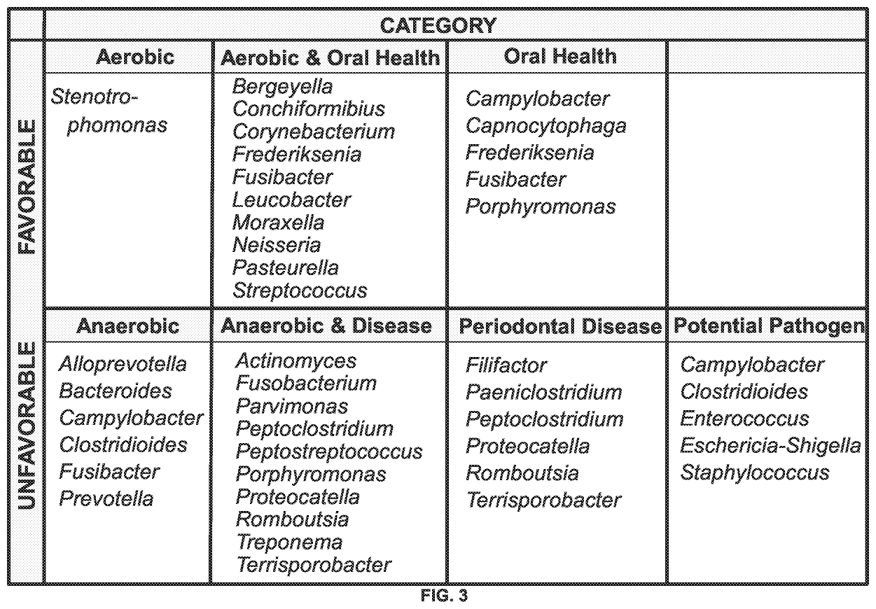

Manipulation of the oral microbiome is accomplished based on measurement of one or more features of the individual's oral microbiome, with interventions selected to adjust the oral microbiome based on the measure. For example, the oral microbiome can be manipulated to improve favorable features, such as an increased incidence of one or more functional types or taxa of microbe, such as taxa associated with good oral health. Also for example, the oral microbiome can be manipulated to limit or eliminate one or more unfavorable features, such as the overabundance of one or more functional types or taxa of microbe, such as known pathogens and/or pathobionts (“pathogen/pathobiont”) or such as those associated with periodontal disease. An example of categories that can be used to define microbiome features is shown in FIG. 3.

The oral health of an individual may be improved by interventions that change the oral microbiome. Oral health may be improved, for example, by interventions such as administration of one or more supplemental compounds, modification of dietary components and/or physical intervention. Such interventions are selected based on one or more features of the individual's oral microbiome. Monitoring or sampling of the oral microbiome can provide a basis for identifying one or more suitable interventions. More generally, monitoring of the oral microbiome can provide a basis for assessing the success of prior interventions, for example, practices associated with dental hygiene, and can serve as a metric for the standard of oral health care.

For example, one or more supplements, dietary components and/or physical interventions can be selected based on a comparison of one or more features of the individual's oral microbiome to the oral microbiomes of another individual or a plurality of individuals. The other individual or the plurality of individuals can be individuals that have good oral health, including for example individuals that do not have one or more particular oral health conditions and/or have low incidence of one or more particular health conditions. The other individual or plurality of individuals can be individuals that have received the one or more supplements, dietary component and/or physical intervention. The other individual or plurality of individuals can be individuals that had one or more particular oral health conditions, received the dietary component and/or one or more of the physical interventions, and thereafter had improved oral health.

The systems and methods of the present invention include obtaining one or more samples from an oral tissue or oral cavity of a mammal, where the one or more samples include microbes of the oral microbiome and/or by-products of the microbes, for example, their DNA, RNA, or metabolic by-products. A sample can be, for example, saliva, tooth material, tissue, plaques, or any combination thereof. It is understood that samples obtained from the oral cavity or oral tissues may include host material in addition to microbes. For example, samples may include mucous secreted by the individual, minerals such as calcium phosphate and iron phosphate, protein, as well as host cells, tissues, DNA and/or RNA.

The one or more samples can be preserved. Plaque and other oral samples can be collected non-invasively using one of various sterile collection devices, such as paper (such as endodontic paper points or specialized cotton-based, cellulose paper designed for sample collection), swabs, or brushes that are gently swiped in different areas of the oral cavity, including the gumline and other plaque retentive surfaces. Samples can be placed in a lysis buffer for DNA extraction shortly after collection, or dried, or samples can be preserved by freezing or placed in a liquid fixative agent, such as ethanol or one of various homemade or commercially available nucleic acid preservation buffers for subsequent analysis.

### Microbiome Features

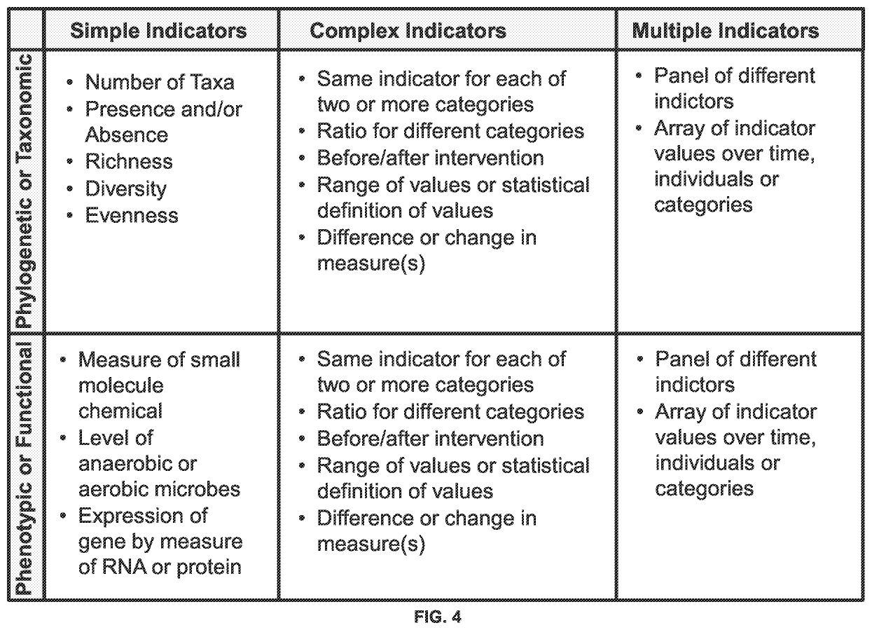

The one or more samples are analyzed to determine one or more microbiome features. As used herein, a “microbiome feature” reflects one or more aspects of the composition or function of microbes in a microbiome, including changes in the composition of microbes or their functional attributes, such as microbial metabolic products. The measure can be, for example and without limitation, a value, a set of values, a range of values, a normalized value, a parameter, or a set of parameters for a statistic, metric, model, or functional relationship, and/or any combination thereof. A microbiome features can reflect one or more aspects of the composition of microbes in the oral microbiome or changes in the composition of microbes in the oral microbiome. The composition of microbes can be defined phylogenetically, for example, based on identification of microbes in a particular class, order, family, genus or species. The composition of microbes can be defined phenotypically, for example, based on respiratory or metabolic requirements. The composition of microbes can be defined both phylogenetically and phenotypically. Phylogenetic identifications of microbes can be used in conjunction with knowledge of the biology of microbial taxa to categorize microbes phenotypically. Exemplary types of measures are shown in FIG. 4.

In some embodiments, the microbiome feature is based, in whole or in part, on the similarity and/or relatedness of microbes, for example, the types or taxa of microbes in the oral microbiome. For example, the microbiome feature can be a range of values for a diversity index based on the taxa of microbes detected in the sample. In some embodiments, the microbiome features is based on alpha diversity indices such as species richness (number of taxa), species evenness (how close in number members of the community are), and/or diversity indices, such as the Shannon Diversity Index, that may incorporate aspects of both richness and evenness.

The microbiome feature can be an identification of the presence/absence or abundance or relative abundance of certain taxa in the sample. For example, the microbiome can be an array of values indicating the taxa present in an oral microbiome, or an array of values indicating for a specified set of taxa whether each is present or absent in an oral microbiome. The microbiome feature can indicate the abundance of microbes, for example, the abundance of each of one or more particular types or taxa of microbes. The abundance of microbes of a particular type or taxa can be expressed in relative terms, for example, relative to the overall abundance of microbes for a sample, for example, as a percentage. Also for example, the microbiome feature can be one or more ratios of the abundance of bacteria in particular phyla, for example, the ratio of Firmicutes to Bacteroidetes, or the ratio of Proteobacteria to Bacteroidetes.

The microbiome feature can reflect or indicate one or more aspects of the function of microbes in the oral microbiome. For example, microbes can be categorized as aerobic and anaerobic, based on their respiratory requirements. In some embodiments, the microbiome feature can be a measure of gene expression, for example, based on RNA and/or proteins, or small molecule chemicals or metabolites produced by the microbiome, such as thiols or sulfur containing amino acids (e.g., cysteine).

Changes in a microbiome feature can indicate a response of the oral microbiome to an intervention or the absence of intervention. For example, a change in alpha diversity, species evenness, diversity, or ratio of phyla can indicate change in the oral health of the individual. For example, a loss of beneficial microbes, an increase in the presence or diversity of potentially harmful microbes, or a loss of overall microbial diversity can indicate a decline in oral health.

The development of periodontal disease can be associated with an expansion of habitat for anaerobic microbes that can live in pockets in the gums. Such metabolic shifts associated with disease progression towards microbes capable of anaerobic respiration can be detected by monitoring. Changes in genes expression and/or protein production can indicate metabolic shifts associated with disease progression, such as anaerobic respiration, protein, amino acid, and sulfur metabolism.

A microbiome feature can be determined for one individual at one time, for example, based on analysis of a sample of the oral microbiome from the oral cavity or oral tissues of the individual. A microbiome feature can be determined for one individual based on analysis of more than one sample of the oral microbiome, where the samples are taken from different surfaces or different types of surfaces in the oral cavity, or from different oral tissues, for example, at about the same time. A microbiome feature can be determined for one individual over a period of time, for example, based on analysis of one or more samples of the oral microbiome from the oral cavity or oral tissues of the individual at one or more different times.

### Reference Samples

A microbiome feature determined for one individual at one time can be compared to a reference measure of the same microbiome feature, or possible microbiome feature, based on a reference sample. Such a comparison can be indicative of oral health, including good oral health and/or an oral health condition or possible oral health condition. Such a comparison can be indicative of likely periodontal disease occurrence or progression. Such a comparison can be indicative of interventions suitable for improving the individual's oral health.

The reference sample can be for the same individual at one or more additional times, for example, based on analysis of one or more additional samples of the oral microbiome from the oral cavity or oral tissue of the individual. The one or more additional samples may be obtained, for example, after an intervention, after a change in the diet of the individual, and/or after an elapse of time, for example, after an elapse of 1 week, 2 weeks, 3 weeks, 4 weeks, 5 weeks, 6 weeks, 7 weeks or 8 weeks, 3 months, 6 months, 9 months, 12 months since a prior sample was obtained. In a preferred embodiment, sampling is repeated at regular intervals, for example, every three to six months, or annually. Such a retesting approach ensures that changes in the individual's oral health are recognized in a timely fashion and can be appropriately addressed, for example, by intervention.

In some embodiments, the frequency of sampling can be increased (i.e. the time between tests made shorter) if sampling of the oral microbiome does not indicate improvement in oral health of an individual. In some embodiments, the type of intervention can be escalated if sampling of the oral microbiome does not indicate improvement in oral health of an individual. For example, an initial intervention can be an at-home intervention, such as a change in diet or oral hygiene practice, and a later intervention can be an intervention performed by a veterinarian or dental professional, such as a dental cleaning.

A microbiome feature determined for one individual, whether at one time, over a period of time, or otherwise, can be compared to reference values for the same microbiome feature, or possible microbiome feature, based on analysis of reference samples obtained from another individual or a plurality of other individuals. The plurality of other individuals can comprise individuals that have good oral health, including for example individuals that do not have one or more particular oral health conditions, individuals that have or had a particular oral health condition, and/or individuals that responded well to one or more dietary component and/or one or more physical interventions.

A microbiome feature determined for one individual can be compared to reference values based on samples from the oral microbiome of that individual at a previous time or times as well as reference values based on analysis of other individuals. For example, a series of reference values and a current sample for an individual can be compared to a series of reference values for a group of other individuals undergoing a similar sequence of interventions.

In a preferred embodiment, the reference values for the microbiome feature are determined using methods similar to the methods used to determine the microbiome feature of the individual's oral microbiome. In some embodiments, the reference values are obtained from a database.

### Methodologies

Measurement of microbiome features can be used to monitor the oral health and/or oral hygiene of an individual, for example, a companion mammal. Measurement of microbiome features can also be used to evaluate the success of interventions, including interventions made for the purpose of altering the microbiome of the individual and/or improving the oral health condition of the individual. Embodiments of the invention encompass identification of interventions to effect changes in the oral microbiome and/or oral health.

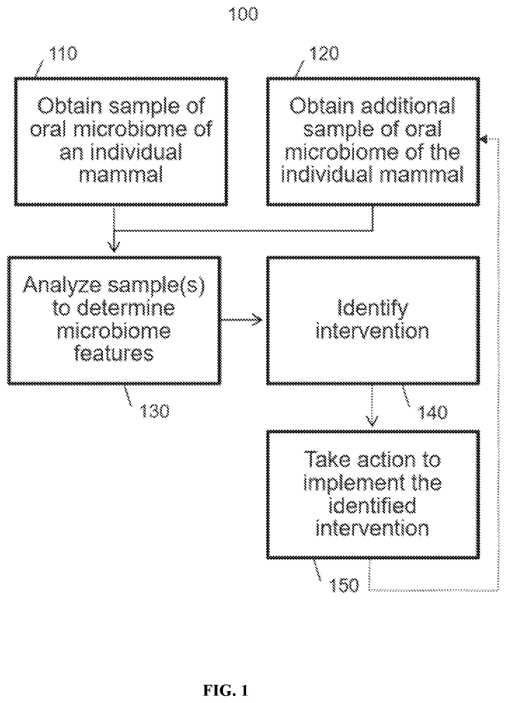

In one embodiment, as shown in FIG. 1, a method 100 begins with the step 110 of obtaining a first sample from an oral tissue or oral cavity of a mammal. The first sample includes microbes of the oral microbiome or the mammal. The first sample can be obtained, for example by rubbing a swab or dental paper along the teeth and gums, or scraping with a dental tool.

The method 100 includes the step 120 of obtaining an additional sample from the oral tissue or oral cavity of the mammal. In a preferred embodiment, the additional sample is obtained in the same manner as the first sample but at a time after the first sample is obtained. For example, the additional sample can be obtained about 1 week, 2 weeks, 3 weeks, 4 weeks, 5 weeks, 6 weeks, 7 weeks, 8 weeks, 3 months, 6 months, 9 months, or 12 months after the first sample is obtained. In alternative embodiments, the additional sample can be obtained at about the same time that the first sample is obtained, for example, in succession during the same encounter with the individual, on the same day, or within 1 week. The additional sample can be obtained in a different manner than the first, for example, using a different collection technique. The first sample can be obtained from one tissue or location in the oral cavity and the additional sample is obtained from a different tissue or location in the oral cavity.

In some embodiments, a preliminary intervention is made between step 110 and step 120. For example, after the first sample is obtained, regular tooth brushing may be started until the time that the additional sample is obtained. Another example is starting daily administration of an oral probiotic designed to introduce healthy oral biofilm organisms after the first sample is obtained. Other examples of interventions are using antibiotics or bacteriophages to target specific bacterial pathogens.

In step 130 of method 100, the first and second samples are analyzed to determine one or more microbiome features of the oral microbiome of the mammal from which the samples were obtained, for example, as discussed previously. For example, samples may be analyzed for changes in the taxonomic diversity of and or metabolites produced by disease associated microbes relative to the taxonomic diversity of and or metabolites produced by health associated microbes.

In step 140 of method 100, an intervention that may cause an adjustment of the oral microbiome, and potentially also the oral health of the individual, is identified. For example, a supplement that can be added to the diet of the mammal is identified. Also for example, a dietary change can be identified, such as reducing sugar in the diet or adding fiber to the diet. The fiber can be a type that will increase the relative abundance of beneficial microbes in the oral microbiome by reducing the availability of such metabolic compounds as amino acids and simple sugars associated with anaerobic respiration in the oral biofilm. The supplement can comprise proteins and/or chelating agents that bind sugars and/or heme and/or iron or heme-containing proteins, for example, to restrict pathogen growth and virulence.

In step 150 of method 100, action is taken to implement the identified intervention. For example, the supplement is administered to the individual or the new dietary component is provided.

Steps 120, 130, 140 and 150 of method 100 can be repeated one or more times for the mammal. For example, about 3 weeks, 4 weeks, 5 weeks, 6 weeks, 7 weeks, 8 weeks, 3 months, 6 months, 9 months, or 12 months after the second sample is obtained, the step 130 of obtaining an additional sample can be repeated. Thereafter, in step 140 of method 100, a further intervention that may cause an adjustment of the oral microbiome, and potentially also the oral health of the individual, is identified. The new intervention can be the same as or different than the intervention identified previously. In step 150, action is again taken to implement the identified new intervention, as described previously.

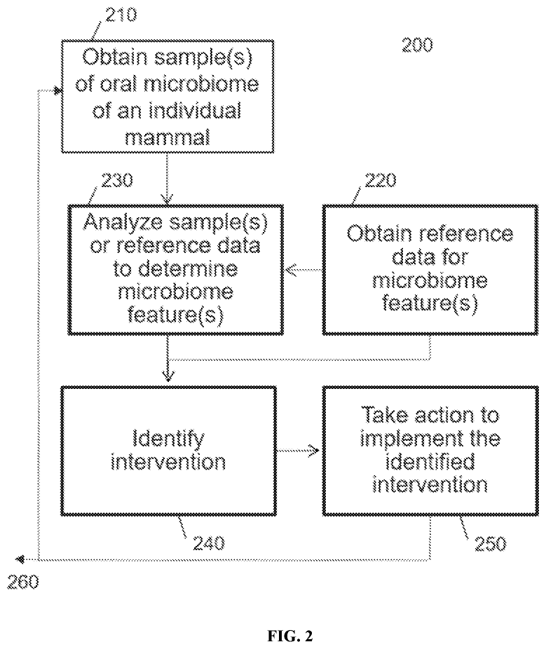

In another embodiment, as shown in FIG. 2, a method 200 begins with the step 210 of obtaining a sample from an oral tissue or oral cavity of a mammal. The sample includes microbes of the oral microbiome of the mammal. The first sample can be obtained, for example, as described with respect to step 110 of FIG. 1.

The method 200 includes the step 220 of obtaining reference data for a plurality of individuals. The reference data can be based on samples from the oral tissues or oral cavities of another individual or a plurality of other individuals. The samples for the plurality of other individuals can be obtained in the same manner as used to obtain a sample in step 210, or in a different manner, for example, using a different collection technique. In one embodiment, the reference data include one or more microbiome features of the plurality of individuals. In an alternative embodiment, the reference data are analyzed to determine one or more microbiome features in step 230.

Also in step 230 of method 200, the sample is analyzed to determine one or more microbiome features of the oral microbiome of the mammal from which the sample was obtained, for example, as discussed previously. In an alternative embodiment, the sample and the reference data are analyzed together to determine one or more microbiome features.

In step 240 of method 200, an intervention that may cause an adjustment of the oral microbiome, and potentially also the oral health of the individual, is identified, for example, as discussed with respect to step 140 of method 100. In step 250 of method 100, action is taken to implement the identified intervention, for example, as discussed with respect to step 150 of method 100.

In one embodiment, after the change in step 250 is made, for example, about 3 weeks, 4 weeks, 5 weeks, 6 weeks, 7 weeks, 8 weeks, 3 months, 6 months, 9 months, or 12 months after the change is made, the process can continue 260 with step 110 or 120 of method 100, as described above. In yet another embodiment, after the change in step 250 of method 200 is made, for example, about 3 weeks, 4 weeks, 5 weeks, 6 weeks, 7 weeks, 8 weeks, 3 months, 6 months, 9 months, or 12 months after the change is made, the process of method 200 can be repeated. The steps 201, 230, and 230 can be repeated. Thereafter, in step 240 of method 200, a new intervention that may cause an adjustment of the oral microbiome is identified. The new condition can be the same as or different than the condition identified previously. In step 250, action is taken to implement the identified new intervention.

### Analysis of Samples

Microbiome features are determined based on data for a plurality of taxa of microbes of the oral microbiome of the individual from which one or more samples were obtained. The one or more samples are analyzed to obtain the data. In one embodiment, a sample or samples are analyzed using sequencing methods known in the art to identify or otherwise characterize the microbes present in each of the samples. Identification includes identification of a particular micro-organism by association to any phylogenetic unit, such as family, genus, or species, or other recognized or meaningful group, as well as identification by other means, such as by analysis of genomic material for particular markers, sequences or other elements. In some embodiments, the micro-organism identification describes the abundance and phylogeny of the microbes at the family level, and in some embodiments, at a level more refined than the family, such as genus, species and/or strain. A taxon can also be defined at a level less refined that the family, for example, class or order.

In some embodiments, the identification of the microbes in a sample or samples is accomplished by shotgun sequencing of extracted DNA and/or by targeted sequencing of microbial diversity, for example (and without limitation), based on the V4 hypervariable region of 16S rRNA. After performing polymerase chain reaction (PCR) for such a marker gene, libraries may be sequenced, for example (and without limitation) using an Illumina MiSeq system, to generate 250 bp (base pair) paired-end amplicon reads, and the amplicon data may be multiplexed using dual barcode combinations for each sample. After sequencing, the samples may be demultiplexed, filtered based on quality scores (FASTQ), and chimeras removed. Binned sequence reads may then be characterized for taxonomic composition (number and abundance), for example and without limitation, by using various software tools such as BLAST that compare sequence reads with reference libraries containing current taxonomic classifications and make de novo assignments for unidentified taxa. The embodiments of the present invention are not limited to the specific sequencing equipment and the specific software and databases described above.

In some embodiments, the microbes in a sample or samples are characterized by chemical analysis of metabolites, for example, volatile sulfur compounds produced by anaerobic bacteria or other metabolites.

In some embodiments, the microbes in a sample or samples are identified by a combination of such methods of analysis of nucleic acids and metabolites. In some embodiments, the microbes in a sample or samples are identified and characterized by analysis of RNA.

Non-limiting embodiments of the invention are enumerated as follows:

Embodiment [0001]: Embodiments of the present invention encompass methods for modifying an oral microbiome of a companion mammal, the method comprising: (a) obtaining a test sample from an oral tissue or oral cavity of the companion mammal, the test sample including microbes of the oral microbiome; (b) analyzing the test sample to obtain test data for a plurality of taxa of the microbes of the oral microbiome of the companion mammal; (c) determining at least one test measure for each of one or more microbiome features based on the test data; (d) obtaining at least one reference measure for each of the one or more microbiome features, the at least one reference measure based on analysis of one or more reference samples from oral tissues or oral cavities of one or more reference companion mammals, the one or more reference samples including microbes of one or more reference oral microbiomes, the analysis of the one or more reference samples providing reference data for a plurality of taxa of microbes, the at least one reference measure based on the reference data; (e) based on the at least one test measure and the at least one reference measure for the one or more microbiome features, identifying a first intervention to modify the oral microbiome of the companion mammal.

Embodiment [0002]: In some embodiments of the present invention, such as, but not limited to, those described in embodiment [0001], the at least one reference measure is based on analysis of one or more reference samples from the oral tissue or oral cavity of the companion mammal.

Embodiment [0003]: In some embodiments of the present invention, such as, but not limited to, those described in embodiment [0001], the at least one reference measure is based on analysis of one or more reference samples from oral tissues or oral cavities of each of two or more reference companion mammals.

Embodiment [0004]: In some embodiments of the present invention, such as, but not limited to, those described in embodiments [0001] and [0003], the reference companion mammals are companion mammals characterized by oral health or periodontal disease state.

Embodiment [0005]: In some embodiments of the present invention, such as, but not limited to, those described in embodiments [0001] to [0004], the test sample is obtained after a preliminary intervention.

Embodiment [0006]: In some embodiments of the present invention, such as, but not limited to, those described in embodiment [0005], at least one of the one or more reference samples is obtained before the preliminary intervention.

Embodiment [0007]: In some embodiments of the present invention, such as, but not limited to, those described in embodiments [0005] and [0006], at least one of the one or more reference samples is obtained after the preliminary intervention.

Embodiment [0008]: In some embodiments of the present invention, such as, but not limited to, those described in embodiment [0005] and [0006], the one or more reference samples are obtained after the preliminary intervention.

Embodiment [0009]: In some embodiments of the present invention, such as, but not limited to, those described in embodiments [0005] to [0008], the first intervention is the same as the preliminary intervention.

Embodiment [0010]: In some embodiments of the present invention, such as, but not limited to, those described in embodiments [0005] to [0008], the first intervention is different from the preliminary intervention.

Embodiment [0011]: In some embodiments of the present invention, such as, but not limited to, those described in embodiments [0005] to [0008], the first intervention is in addition to the preliminary intervention.

Embodiment [0012]: In some embodiments of the present invention, such as, but not limited to, those described in embodiments [0001] to [0011] and [0015] to [0037], the one or more microbiome features comprise an indicator of taxonomic richness or diversity for each of two or more categories of microbes.

Embodiment [0013]: In some embodiments of the present invention, such as, but not limited to, those described in embodiment [0012], the one or more microbiome features comprise a ratio of taxonomic richness or diversity for periodontal disease-associated microbes to taxonomic richness or diversity for oral health-associated microbes.

Embodiment [0014]: In some embodiments of the present invention, such as, but not limited to, those described in embodiments [0001] to [0011] and [0015] to [0037], the reference data provides a threshold value for assessing the at least one test measure for each of one or more microbiome features.

Embodiment [0015]: In some embodiments of the present invention, such as, but not limited to, those described in embodiments [0001] to [0014], the first intervention is selected from administration of a probiotic, prebiotic fiber, prebiotic supplement, bacteriophage, bacteriophage-associated protein, bacteriophage lysins, antimicrobial, antimicrobial peptide, nanoparticles with biocidal properties, sodium bicarbonate, chelating agents, glycoproteins, phototherapy, and any combination thereof.

Embodiment [0016]: In some embodiments of the present invention, such as, but not limited to, those described in embodiments [0001] to [0014], the first intervention comprises administration of a chelating agent and prebiotics.

Embodiment [0017]: In some embodiments of the present invention, such as, but not limited to, those described in embodiments [0001] to [0014], the first intervention comprises modification of a dietary component.

Embodiment [0018]: In some embodiments of the present invention, such as, but not limited to, those described in embodiments [0001] to [0014], the first intervention is selected from tooth brushing, tooth flossing, mechanical removal or reduction in biofilm, dental cleaning, tooth extraction, root canal, dental examination, administration of an antibiotic, and any combinations thereof.

Embodiment [0019]: In some embodiments of the present invention, such as, but not limited to, those described in embodiments [0005] to [0011], the preliminary intervention is selected from administration of a probiotic, prebiotic fiber, prebiotic supplement, bacteriophage, bacteriophage-associated protein, bacteriophage lysins, antimicrobial, antibiotic, antimicrobial peptide, nanoparticles with biocidal properties, sodium bicarbonate, chelating agents, glycoproteins, phototherapy, and any combination thereof.

Embodiment [0020]: In some embodiments of the present invention, such as, but not limited to, those described in embodiments [0005] to [0011], the preliminary intervention comprises administration of a chelating agent and prebiotics.

Embodiment [0021]: In some embodiments of the present invention, such as, but not limited to, those described in embodiments [0005] to [0011], the preliminary intervention comprises modification of a dietary component.

Embodiment [0022]: In some embodiments of the present invention, such as, but not limited to, those described in paragraph embodiments [0005] to [0011] and [0019] to [0021], the preliminary intervention is selected from tooth brushing, tooth flossing, mechanical removal or reduction in biofilm, dental cleaning, tooth extraction, root canal, dental examination and any combinations thereof.

Embodiment [0023]: In some embodiments of the present invention, such as, but not limited to, those described in embodiment [0005], the first intervention includes assessing the success of the preliminary intervention.

Embodiment [0024]: In some embodiments of the present invention, such as, but not limited to, those described in embodiment [0023], success of the preliminary intervention is indicated by one or more of an increase in aerobic bacteria, an increase in oral-health associated bacteria, a decrease in anaerobic bacteria and a decrease in periodontal disease-associated bacteria.

Embodiment [0025]: In some embodiments of the present invention, such as, but not limited to, those described in embodiment [0023], success of the preliminary intervention is indicated by a decrease in pathogen/pathobiont bacteria.

Embodiment [0026]: In some embodiments of the present invention, such as, but not limited to, those described in embodiment [0023], assessing the success of the preliminary intervention includes assessing whether there is an improvement in a measure of microbes in at least one category.

Embodiment [0027]: In some embodiments of the present invention, such as, but not limited to, those described in embodiment [0026], the at least one category is selected from aerobic bacteria, anaerobic bacteria, periodontal-associated bacteria, pathogen/pathobiont bacteria, and oral-health associated bacteria.

Embodiment [0028]: In some embodiments of the present invention, such as, but not limited to, those described in embodiment [0023], assessing the success of the preliminary intervention includes determining one or more of an increase in taxonomic diversity, taxonomic richness, and taxonomic evenness.

Embodiment [0029]: In some embodiments of the present invention, such as, but not limited to, those described in embodiments [0023] to [0028], assessing the success of the preliminary intervention includes consideration of more or more of the companion mammal's diet, general health, exposure to microbes, dental care history, and oral hygiene.

Embodiment [0030]: In some embodiments of the present invention, such as, but not limited to, those described in embodiment [0029], oral hygiene comprises one or more of health of teeth, health of gums, health of tongue, low or no plaque and tartar, periodontal health, and fresh breath.

Embodiment [0031]: In some embodiments of the present invention, such as, but not limited to, those described in embodiments [0001] to [0030], the method further comprises identifying a second intervention.

Embodiment [0032]: In some embodiments of the present invention, such as, but not limited to, those described in embodiment [0032], the second intervention is selected from administration of a probiotic, prebiotic fiber, prebiotic supplement, bacteriophage, bacteriophage-associated protein, bacteriophage lysins, antimicrobial, narrow-spectrum antibiotic, antimicrobial peptide, nanoparticles with biocidal properties, sodium bicarbonate, chelating agents, glycoproteins, phototherapy, and any combination thereof.

Embodiment [0033]: In some embodiments of the present invention, such as, but not limited to, those described in embodiment [0030], the second intervention is selected from tooth brushing, tooth flossing, mechanical removal or reduction in biofilm, dental cleaning, tooth extraction, root canal and dental examination.

Embodiment [0034]: In some embodiments of the present invention, such as, but not limited to, those described in embodiment [0030], the second intervention is the same as the first intervention.

Embodiment [0035]: In some embodiments of the present invention, such as, but not limited to, those described in embodiment [0030], the first intervention is determined to be unsuccessful.

Embodiment [0036]: In some embodiments of the present invention, such as, but not limited to, those described in embodiment [0034], the second intervention is different than the first intervention.

Embodiment [0037]: In some embodiments of the present invention, such as, but not limited to, those described in embodiments [0001] to [0035], the at least one test measure and the at least one reference measure each comprise a value, a set of values, a range of values, a normalized value, a parameter or a set of parameters for a statistic, metric, or model, or any combination thereof.

Embodiment [0038]: In some embodiments of the present invention, such as, but not limited to, those described in embodiments [0001] to [0037], the at least one test measure and the at least one reference measure each comprise one or more indicators of taxonomic diversity, taxonomic richness, and taxonomic evenness. OTHER?

Embodiment [0039]: In some embodiments of the present invention, such as, but not limited to, those described in embodiments [0001] to [0037], the one or more microbiome features comprises an indication of similarity and/or relatedness of two or more of the plurality of taxa of the microbes of the oral microbiome.

Embodiment [0040]: In some embodiments of the present invention, such as, but not limited to, those described in embodiments [0001] to [0037], the one or more microbiome features reflect one or more aspects of the composition of microbes in the oral microbiome or changes in the composition of microbes in the oral microbiome.

Embodiment [0041]: In some embodiments of the present invention, such as, but not limited to, those described in embodiments [0001] to [0037], the one or more microbiome features comprise one or more measures of relative abundance of one or more of the plurality of taxa of the microbes of the oral microbiome of the companion mammal.

Embodiment [0042]: In some embodiments of the present invention, such as, but not limited to, those described in embodiments [0001] to [0037], the at least one test measure and the at least one reference measure for each of the one or more microbiome features indicates presence of two or more of the plurality of taxa, or presence or absence of two or more of a plurality of predefined taxa.

Embodiment [0043]: In some embodiments of the present invention, such as, but not limited to, those described in embodiments [0001] to [0037], the one or more microbiome features reflect one or more aspects of the function of microbes in the oral microbiome or changes in the microbial metabolic products associated with a microbiome.

Embodiment [0044]: In some embodiments of the present invention, such as, but not limited to, those described in embodiment [0043], the one or more microbiome features indicate activity for one or more metabolic categories of microbes in the oral microbiome.

Embodiment [0045]: In some embodiments of the present invention, such as, but not limited to, those described in embodiment [0044], the one or more microbiome features comprise an indicator of anaerobic bacteria and an indicator of aerobic bacteria.

Embodiment [0046]: In some embodiments of the present invention, such as, but not limited to, those described in embodiments [0001] to [0037], the one or more microbiome features comprise an indicator of periodontal disease-associated microbes or an indicator of pathogen/pathobiont bacteria.

Embodiment [0047]: In some embodiments of the present invention, such as, but not limited to, those described in embodiments [0001] to [0037], the one or more microbiome features comprise an indicator of periodontal disease-associated bacteria and an indicator of oral health-associated bacteria.

Embodiment [0048]: In some embodiments of the present invention, such as, but not limited to, those described in embodiments [0001] to [0037], the one or more microbiome features comprise indicators for two or more of anaerobic bacteria, aerobic bacteria, periodontal disease-associated bacteria, oral-health-associated bacteria, and pathogen/pathobiont bacteria.

Embodiment [0049]: In some embodiments of the present invention, such as, but not limited to, those described in embodiments [0001] to [0037], the one or more microbiome features comprise indicators for three or more of anaerobic bacteria, aerobic bacteria, periodontal disease-associated bacteria, oral-health-associated bacteria, and pathogen/pathobiont bacteria.

Embodiment [0050]: In some embodiments of the present invention, such as, but not limited to, those described in embodiments [0001] to [0037], the one or more microbiome features further comprises a measure of two or more taxa of microbes.

Embodiment [0051]: In some embodiments of the present invention, such as, but not limited to, those described in embodiments [0001] to [0037], each of the one or more microbiome features comprises and a presence, an amount, or a relative abundance of microbes according to taxa or category.

Embodiment [0052]: In some embodiments of the present invention, such as, but not limited to, those described in embodiments [0001] to [0037], the one or more microbiome features indicate a change in presence, amount, or relative abundance of microbes according to taxa or category.

Embodiment [0053]: In some embodiments of the present invention, such as, but not limited to, those described in embodiments [0001] to [0037], each of the one or more microbiome features indicates the relative abundance or a change in relative abundance of aerobic bacteria, anaerobic bacteria, periodontal disease-associated and pathogen/pathobiont bacteria.

Embodiment [0055]: In some embodiments of the present invention, such as, but not limited to, those described in embodiments [0001] to [0053], the analyzing comprises analyzing genetic material in the test sample and each of the one or more reference samples.

Embodiment [0055]: In some embodiments of the present invention, such as, but not limited to, those described in embodiment [0054], the analyzing further comprises identifying the plurality of taxa of microbes by sequence analysis.

Embodiment [0056]: In some embodiments of the present invention, such as, but not limited to, those described in embodiment [0055], identifying the plurality of taxa of microbes is accomplished by shotgun sequencing of extracted deoxyribonucleic acid (DNA) and/or by targeted sequencing of bacterial diversity.

Embodiment [0057]: In some embodiments of the present invention, such as, but not limited to, those described in embodiment [0054], the genetic material includes ribonucleic acid (RNA) from microbes of the oral microbiome of the companion mammal.

Embodiment [0058]: In some embodiments of the present invention, such as, but not limited to, those described in embodiment [0054], the genetic material includes deoxyribonucleic acid (DNA) from microbes of the oral microbiome of the companion mammal.

Embodiment [0059]: In some embodiments of the present invention, such as, but not limited to, those described in embodiments [0001] to [0053], the analyzing comprises analyzing proteins in the test sample and each of the one or more reference samples.

Embodiment [0060]: In some embodiments of the present invention, such as, but not limited to, those described in embodiments [0001] to [0059], the analyzing further comprises characterization of volatile metabolites using chemical analysis.

Embodiment [0061]: In some embodiments of the present invention, such as, but not limited to, those described in embodiments [0054] to [0060], the analyzing further comprises characterizing the composition or metabolic function of a biofilm in the oral microbiome of the companion mammal.

Embodiment [0062]: In some embodiments of the present invention, such as, but not limited to, those described in embodiments [0001] to [0061], the method further comprises providing a plan for the companion mammal's oral health.

Embodiment [0063]: In some embodiments of the present invention, such as, but not limited to, those described in embodiments [0001] to [0061], the method further comprises implementing the first intervention and modifying the oral microbiome of the companion mammal.

Embodiment [0064]: In some embodiments of the present invention, such as, but not limited to, those described in embodiment [0063], modifying the oral microbiome of the companion mammal comprises improving at least one of the one or more microbiome feature of the oral microbiome of the companion mammal.

Embodiment [0065]: In some embodiments of the present invention, such as, but not limited to, those described in embodiments [0001] to [0061], the method further comprises providing materials for executing the first intervention.

Embodiment [0066]: In some embodiments of the present invention, such as, but not limited to, those described in embodiments [0001] to [0061], the first intervention modifies the oral microbiome of the companion mammal by improving the balance of microbes of the oral microbiome, by increasing favorable microbes, and/or by decreasing unfavorable microbes.

Embodiment [0067]: In some embodiments of the present invention, such as, but not limited to, those described in embodiments [0001] to [0061], the method further comprises preserving the test sample.

Embodiment [0068]: In some embodiments of the present invention, such as, but not limited to, those described in embodiment [0067], preserving the test sample includes one or more of drying the test sample, placing the test sample in a buffer, and combining the test sample with a preservative.

Embodiment [0069]: Embodiments of the present invention encompass a method of preparing oral microbiome samples of a companion mammal for identification of an intervention to modify the oral microbiome of the companion mammal, the method comprising: (a) obtaining a test sample from an oral tissue or oral cavity of the companion mammal, the test sample including microbes of the oral microbiome of the companion mammal; (b) preserving one or more nucleic acids from the test sample; (c) obtaining at least one reference measure for each of the one or more microbiome features, the at least one reference measure based on analyzing nucleic acids from one or more reference samples from oral tissues or oral cavities of one or more reference companion mammals, the one or more reference samples including microbes of one or more reference oral microbiomes, the analysis of the nucleic acids from the one or more reference samples providing reference data for a plurality of taxa of microbes, the at least one reference measure based on the reference data; (d) analyzing nucleic acids from the test sample to obtain test data for a plurality of taxa of the microbes of the oral microbiome of the companion mammal; (e) determining at least one test measure for each of one or more microbiome features based on the test data and the at least one reference measure.

Embodiment [0070]: In some embodiments of the present invention, such as, but not limited to, those described in embodiment [0069], preserving one or more nucleic acids from the test sample comprises one or more of drying the sample, placing the test sample in a buffer, and combining the test sample with a preservative.

Embodiment [0071]: In some embodiments of the present invention, such as, but not limited to, those described in embodiment [0070], the buffer comprises ethanol, NAP, or CD1

Embodiment [0072]: In some embodiments of the present invention, such as, but not limited to, those described in embodiment [0069], analyzing nucleic acids from the test sample or one of the one or more reference samples comprises extracting DNA, extracting RNA, amplifying nucleic acid sequences in the test sample,

Embodiment [0073]: Embodiments of the present invention encompass a method of assessing an oral microbiome sample from a companion mammal comprising: (a) obtaining a test sample from an oral tissue or cavity of the companion mammal, the test sample including microbes of the oral microbiome; (b) preparing an assay sample comprising nucleic acid from the test sample; (c) sequencing a portion of the nucleic acid to identify microbes in the test sample according to two or more taxa; (d) identifying the presence and/or level of microbes for each of the two or more taxa; (e) assessing the test sample based upon the presence and/or level of microbes for each of the two or more taxa according to one or more categories of microbes.

Embodiment [0074]: In some embodiments of the present invention, such as, but not limited to, those described in embodiment [0073], the one or more categories are selected from anaerobic, aerobic, periodontal disease-associated, oral health associated, and potential pathogen/pathobiont.

Embodiment [0075]: In some embodiments of the present invention, such as, but not limited to, those described in embodiment [0073], the method further comprises identifying a plan for the companion's oral health based on the assessment.

Embodiment [0076]: In some embodiments of the present invention, such as, but not limited to, those described in embodiments [0001] to [0075], the companion mammal is a dog.

Embodiment [0077]: In some embodiments of the present invention, such as, but not limited to, those described in embodiments [0001] to [0075], the companion mammal is a cat.

Embodiment [0078]: In some embodiments of the present invention, such as, but not limited to, those described in embodiments [0001] to [0075], the companion mammal and the one or more reference companion mammals are of a same species.

Embodiment [0079]: In some embodiments of the present invention, such as, but not limited to, those described in embodiment [0074], the companion mammal and at least one of the one or more reference companion mammals are of a same breed.

Embodiment [0080]: In some embodiments of the present invention, such as, but not limited to, those described in embodiment [0075], the companion mammal and at least one of the one or more reference companion mammals are of different breeds.

Embodiment [0081]: In some embodiments of the present invention, such as, but not limited to, those described in embodiments [0001] to [0075], the method further comprises providing materials for obtaining the test sample.

### EXAMPLES

The following examples are given to aid in understanding the invention, but it is to be understood that the invention is not limited to the particular materials or procedures of the examples.

### Example 1: Change in Availability of Iron

Bacteria in the oral microbiome require iron and differ in their ability to obtain it from their environment. Lactoferrin and transferrin are proteins in saliva and blood that bind iron, reducing the amount available to oral bacteria. However, some bacteria are able to subvert such host defenses attempting to restrict access to iron because they are able to utilize the iron in these host glycoproteins (Banerjee et al. 2012). For example, bacteria in the families Neisseriaceae and Pasteurellaceae are able to make use of iron bound within transferrin as well as iron bound within lactoferrin (Ratledge and Dover 2000). Such bacteria include Neisseria shayeganii and Pasteurella dagmatis (Davis et al. 2013; Holcombe et al. 2014). Also, P. cangingivalis may dominate in healthy biofilms in canines due to its ability to synthesize its own heme (O'Flynn et al. 2015). The oral microbiome can be manipulated by restricting the availability of iron to pathogenic bacteria associated with periodontal disease through the use of chelating agents that make iron as well as molecules containing iron less accessible. For example, by introducing glycoproteins that make the iron bound by them inaccessible to pathogens, the abundance of pathogenic bacteria in the oral microbiome can be reduced.

### Example 2: Change in Availability of Sugar

Bacteria in the oral microbiome require sugar, and their abundance may be reduced and/or regulated if growth rates are kept sufficiently low. One strategy for reducing growth rates is to reduce the availability of sugars, for example, by replacing traditional sugars with sugar alcohols such as xylitol and maltitol that are nonfermentable. Dental chews, chewing gum, tooth brushing, mouthwash, and chelating agents can also be used to remove sugars from the oral cavity. The oral microbiome can be manipulated by administering one or more such dietary components. This may reduce the volume of dental plaque, prevent decreases in plaque pH, stimulate saliva production, and promote selection of less virulent, sugar alcohol-resistant bacteria in the oral biofilm, particularly Streptococcus (Würsch and Koellreutter 1982; Dhar and Tinanoff 2017).

### Example 3a: Medical Intervention

Once periodontal disease progresses beyond a certain point, medical intervention may be necessary to remove calculus and the associated biofilms that trigger inflammation and bleeding in the subgingival environment.

Testing for the change in the abundance of certain microorganisms, including bacteria, fungi and protists, before and after various interventions can indicate when additional examination and dental work are necessary. A reduction in the prevalence of anaerobic bacteria including Bacteroides, Peptostreptococcus, Porphyromonas, and Prevotella indicates a positive response to the intervention. Likewise, increases in the prevalence of aerobic bacteria, including Streptococcus, Pasteurella, Moraxella, Neisseria and Bergeyella also indicate a positive response to the intervention. As a result, a positive response to an oral health intervention should be correlated with an increase in the ratio of Firmicutes to Bacteroidetes in the oral biofilm associated with the gumline. Increased bacterial diversity, richness and evenness, are also associated with a favorable response.

The after sample is compared to the first sample to assess the effectiveness of the medical intervention. If the second sample falls below a reference range, this may indicate that escalating targeted interventions, such as additional dental cleaning, antibiotics, root canal, and tooth extractions or removal may be necessary.

TABLE 1

| | Chihuahua mix | | Border collie mix | | Golden retriever mix | |
|---|---|---|---|---|---|---|
| Taxonomy | Before | After | Before | After | Before | After |
| k: Bacteria; | | | | | | |
| p: Actinobacteria; | | | | | | |
| c: Actinobacteria; | | | | | | |
| o: Actinomycetales; | | | | | | |
| f: Actinomycetaceae; | | | | | | |
| g: Actinomyces | 0.035 | 0.042 | 0.002 | 0.003 | 0.014 | 0.024 |
| k: Bacteria; | | | | | | |
| p; Actinobacteria; | | | | | | |
| c: Actinobacteria; | | | | | | |
| o: Corynebacteriales; | | | | | | |
| f: Corynebacteriaceae; | | | | | | |
| g: Corynebacterium | 0.001 | 0.001 | 0.001 | 0.011 | 0.002 | 0.016 |
| k: Bacteria; | | | | | | |
| p: Bacteroidetes; | | | | | | |
| c: Bacteroidia; | | | | | | |
| o: Bacteroidales; | | | | | | |
| f: Bacteroidaceae; | | | | | | |
| g: Bacteroides | 0.011 | 0.014 | 0.016 | 0.002 | 0.037 | 0.001 |
| k: Bacteria; | | | | | | |
| p: Bacteroidetes; | | | | | | |
| c: Bacteroidia; | | | | | | |
| o: Bacteroidales; | | | | | | |
| f: Porphyromonadaceae; | | | | | | |
| g: Porphyromonas | 0.181 | 0.281 | 0.168 | 0.077 | 0.423 | 0.098 |
| k: Bacteria; | | | | | | |
| p: Bacteroidetes; | | | | | | |
| c: Bacteroidia; | | | | | | |
| o: Bacteroidales; | | | | | | |
| f: Prevotellaceae; | | | | | | |
| g: Alloprevotella | 0.158 | 0.114 | 0.123 | 0.002 | 0.045 | 0.001 |
| k: Bacteria; | | | | | | |
| p: Bacteroidetes; | | | | | | |
| c: Bacteroidia; | | | | | | |
| o: Bacteroidales; | | | | | | |
| f: Prevotellaceae; | | | | | | |
| g: Prevotella 7 | 0.004 | 0.013 | 0.000 | 0.000 | 0.003 | 0.000 |
| k: Bacteria; | | | | | | |
| p: Bacteroidetes; | | | | | | |
| c: Bacteroidia; | | | | | | |
| o: Bacteroidales; | | | | | | |
| f: Prevotellaceae; | | | | | | |
| g: Prevotellaceae | 0.005 | 0.023 | 0.000 | 0.000 | 0.008 | 0.000 |
| UCG-003 | | | | | | |
| k: Bacteria; | | | | | | |
| p: Bacteroidetes; | | | | | | |
| c: Bacteroidia; | | | | | | |
| o: Flavobacteriales; | | | | | | |
| f: Flavobacteriaceae; | | | | | | |
| g: Capnocytophaga | 0.008 | 0.009 | 0.065 | 0.012 | 0.009 | 0.008 |
| k: Bacteria; | | | | | | |
| p: Bacteroidetes; | | | | | | |
| c: Bacteroidia; | | | | | | |
| o: Flavobacteriales; | | | | | | |
| f: Weeksellaceae; | | | | | | |
| g: Bergeyella | 0.044 | 0.040 | 0.022 | 0.022 | 0.008 | 0.040 |
| k: Bacteria; | | | | | | |
| p: Firmicutes; | | | | | | |
| c: Clostridia; | | | | | | |
| o: Clostridiales; | | | | | | |
| f: Defluviitaleaceae; | | | | | | |
| g: Defluviitaleaceae | 0.017 | 0.012 | 0.001 | 0.003 | 0.015 | 0.028 |
| UCG-011 | | | | | | |
| k: Bacteria; | | | | | | |
| p: Firmicutes; | | | | | | |
| c: Clostridia; | | | | | | |
| o: Clostridiales; | | | | | | |
| f: Family XI; | | | | | | |
| g: Helcococcus | 0.000 | 0.000 | 0.000 | 0.000 | 0.004 | 0.002 |
| k: Bacteria; | | | | | | |
| p: Firmicutes; | | | | | | |
| c: Clostridia; | | | | | | |
| o: Clostridiales; | | | | | | |
| f: Family XII; | | | | | | |
| g: Fusibacter | 0.003 | 0.004 | 0.004 | 0.002 | 0.011 | 0.006 |
| k: Bacteria; | | | | | | |
| p: Firmicutes; | | | | | | |
| c: Clostridia; | | | | | | |
| o: Clostridiales; | | | | | | |
| f: Peptococcaceae; | | | | | | |
| g: Peptococcus | 0.013 | 0.007 | 0.002 | 0.001 | 0.013 | 0.004 |
| k: Bacteria; | | | | | | |
| p: Firmicutes; | | | | | | |
| c: Clostridia; | | | | | | |
| o: Clostridiales; | | | | | | |
| f: Peptostreptococcaceae; | | | | | | |
| g: Proteocatella | 0.001 | 0.001 | 0.003 | 0.010 | 0.005 | 0.039 |
| k: Bacteria; | | | | | | |
| p: Fusobacteria; | | | | | | |
| c: Fusobacteriia; | | | | | | |
| o: Fusobacteriales; | | | | | | |
| f: Fusobacteriaceae; | | | | | | |
| g: Fusobacterium | 0.044 | 0.043 | 0.004 | 0.010 | 0.055 | 0.021 |
| k: Bacteria; | | | | | | |
| p: Fusobacteria; | | | | | | |
| c: Fusobacteriia; | | | | | | |
| o: Fusobacteriales; | | | | | | |
| f: Leptotrichiaceae; | | | | | | |
| g: Oceanivirga | 0.067 | 0.043 | 0.000 | 0.000 | 0.000 | 0.000 |
| k: Bacteria; | | | | | | |
| p: Proteobacteria; | | | | | | |
| c: Gammaproteobacteria; | | | | | | |
| o: Betaproteobacteriales; | | | | | | |
| f: Aquaspirillaceae; | | | | | | |
| g: Aquaspirillum | 0.002 | 0.000 | 0.056 | 0.000 | 0.000 | 0.000 |
| k: Bacteria; | | | | | | |
| p: Proteobacteria; | | | | | | |
| c: Gammaproteobacteria; | | | | | | |
| o: Betaproteobacteriales; | | | | | | |
| f: Burkholderiaceae; | | | | | | |
| g: Lautropia | 0.001 | 0.005 | 0.010 | 0.016 | 0.001 | 0.002 |
| k: Bacteria; | | | | | | |
| p; Proteobacteria; | 0.004 | 0.004 | 0.017 | 0.137 | 0.030 | 0.198 |
| c: Gammaproteobacteria; | | | | | | |
| o: Betaproteobacteriales; | | | | | | |
| f: Neisseriaceae; | | | | | | |
| g: Conchiformibius | | | | | | |
| k: Bacteria; | | | | | | |
| p; Proteobacteria; | | | | | | |
| c: Gammaproteobacteria; | | | | | | |
| o: Betaproteobacteriales; | | | | | | |
| f: Neisseriaceae; | | | | | | |
| g: Neisseria | 0.061 | 0.040 | 0.002 | 0.018 | 0.002 | 0.012 |
| k: Bacteria; | | | | | | |
| p; Proteobacteria; | | | | | | |
| c: Gammaproteobacteria; | | | | | | |
| o: Cardiobacteriales; | | | | | | |
| f: Cardiobacteriaceae; | | | | | | |
| g: uncultured | 0.001 | 0.001 | 0.001 | 0.023 | 0.001 | 0.001 |
| k: Bacteria; | | | | | | |
| p; Proteobacteria; | | | | | | |
| c: Gammaproteobacteria; | | | | | | |
| o: Pasteurellales; | | | | | | |
| f: Pasteurellaceae; | | | | | | |
| g: [none] | 0.004 | 0.004 | 0.007 | 0.002 | 0.012 | 0.008 |
| k: Bacteria; | | | | | | |
| p; Proteobacteria; | | | | | | |
| c: Gammaproteobacteria; | | | | | | |
| o: Pasteurellales; | | | | | | |
| f: Pasteurellaceae; | | | | | | |
| g: Frederiksenia | 0.008 | 0.003 | 0.019 | 0.147 | 0.050 | 0.254 |
| k: Bacteria; | | | | | | |
| p; Proteobacteria; | | | | | | |
| c: Gammaproteobacteria; | | | | | | |
| o: Pasteurellales; | | | | | | |
| f: Pasteurellaceae; | | | | | | |
| g: Haemophilus | 0.022 | 0.008 | 0.001 | 0.087 | 0.009 | 0.024 |
| k: Bacteria; | | | | | | |
| p; Proteobacteria; | | | | | | |
| c: Gammaproteobacteria; | | | | | | |
| o: Pasteurellales; | | | | | | |
| f: Pasteurellaceae; | | | | | | |
| g: Pasteurella | 0.087 | 0.097 | 0.004 | 0.004 | 0.015 | 0.079 |
| k: Bacteria; | | | | | | |
| p; Proteobacteria; | | | | | | |
| c: Gammaproteobacteria; | | | | | | |
| o: Pseudomonadales; | | | | | | |
| f: Moraxellaceae; | | | | | | |
| g: Moraxella | 0.061 | 0.056 | 0.350 | 0.158 | 0.064 | 0.003 |
| k: Bacteria; | | | | | | |
| p; Proteobacteria; | | | | | | |
| c: Gammaproteobacteria; | | | | | | |
| o: Xanthomonadales; | 0.003 | 0.002 | 0.003 | 0.020 | 0.001 | 0.001 |
| f: Xanthomonadaceae; | | | | | | |
| g: Luteimonas | | | | | | |
| k: Bacteria; | | | | | | |
| p; Spirochaetes; | | | | | | |
| c: Spirochaetia; | | | | | | |
| o: Spirochaetales; | | | | | | |
| f: Spirochaetaceae; | | | | | | |
| g: Treponema 2 | 0.015 | 0.030 | 0.048 | 0.004 | 0.060 | 0.001 |
| k: Bacteria; | | | | | | |
| p; Tenericutes; | | | | | | |
| c: Mollicutes; | | | | | | |
| o: Acholeplasmatales; | | | | | | |
| f: Acholeplasmataceae; | | | | | | |
| g: Acholeplasma | 0.000 | 0.022 | 0.002 | 0.000 | 0.009 | 0.000 |

### Example 4: Toothbrushing

The gumline of a domestic dog (Canis lupis familiaris) was tested before and after three weeks of tooth brushing.

As shown in TABLE 1, this six year old, male, Chihuahua mix had high levels of anaerobic bacteria in the genus Porphyromonas at the beginning and end of a three week period. At the beginning of this time, before tooth brushing began, genus-level diversity was 3.01, genus-level richness was 86, and evenness was 0.68. After the first sample, an intervention of regular tooth brushing began on a daily basis using a chicken-flavored toothpaste designed for domestic dogs. After three weeks of tooth brushing, genus-level diversity was 2.85, genus-level richness was 90, and evenness was 0.63.

By comparing these microbiome features before and after three weeks of tooth brushing, it is seen that the relative abundance of Porphyromonas was not reduced and the ratio of Firmicutes to Bacteroidetes decreased from 0.14 to 0.09. These results indicate no improvement in oral health, so a dental examination or another intervention is in order.

### Example 5: Prebiotic Supplement Added to Water Bowl

One quarter of a teaspoon of an oral supplement was added to the water bowl on a daily basis for a two week period. It contained a chelating agent blocking bacterial sugar transport and prebiotics to support the growth of beneficial bacteria in the canine oral microbiome. Samples were collected from two domestic dogs (Canis lupis familiaris) prior to the use of this supplement and two weeks after initiating its use.

As shown in TABLE 1, prior to the addition of the prebiotic product to the water dish, both dogs had high levels of anaerobic bacteria associated with biofilms in the canine oral microbiome, particularly members of the genus Porphyromonas.

Dog 1 was an 8 year old, male, Border Collie mix. After two weeks on the prebiotic supplement, as shown in TABLE 1, the relative abundance of anaerobic bacteria, particularly Porphyromonas and Alloprevotella, was reduced, indicating that the community responded appropriately to the intervention. Genus-level diversity also increased from 2.35 to 2.8 (Shannon Diversity Index), genus-level richness (number of different genera in the sample) increased from 89 to 102, and evenness increased from 0.52 to 0.6. The proportion of Firmicutes to Bacteroidetes was more than 5 times greater in the second sample, increasing from 0.05 to 0.27.

Dog 2 (12 year old, male, Golden Retriever mix): After two weeks on the prebiotic supplement, as shown in TABLE 1, the relative abundance of Porphyromonas and Fusobacterium decreased and the relative abundance of the early colonizer Frederiksenia, Conchiformibius, Pasteurella (facultative anaerobe), and the aerobic bacterium, Begeyella increased, indicating that the community is responding appropriately to the intervention. Alpha diversity responses were more subtle in this dog; genus-level diversity increased slightly from 2.61 to 2.69, evenness increased slightly from 0.57 to 0.61, but genus level richness decreased from 100 to 83. The proportion of Firmicutes to Bacteroidetes increased nearly 6-fold from 0.15 to 0.88.

These results indicate improvement in oral health. Follow-up testing is recommended every three months for both dogs. If the response failed to indicate a beneficial shift in the microbiome community composition and/or function, then additional interventions, including a dental examination may be advisable.

### Example 6: Use of Categories

Example of categories of bacteria used to create diversity metrics to describe the structure and function of the oral biofilm community are shown in Table 2. Disease associations are based on correlations between periodontal disease state and ecological succession of the oral biofilm communities. Tolerance of aerobic and or anaerobic conditions are expectations based on examination of the literature for genomes and growth conditions favored by any isolates obtained for these species. Periodontal disease-associated bacteria (“PD”) tend to require anaerobic growth conditions, health-associated bacteria (“Health”) tend to colonize the oral cavity earlier in life and to favor aerobic conditions, and potential pathogens and/or pathobionts (“Pathobionts”) tend to have flexibility and some are facultatively anaerobic.

TABLE 2

| Genus | Species | Category | Respiration |
|---|---|---|---|
| Actinomyces | canis, coleocanis, | PD | Facultative and |
| | graevenitzii | | Obligate |
| | F0530, hordeovulneris, | | Anaerobes |
| | israelii, | | |
| | sp, COT374, | | |
| | COT404 | | |
| Porphyromonas | gulae, gingivicanis | PD | Anaerobic |
| Parvimonas | micra | PD | Anaerobic |
| Peptostreptococcus | spp | PD | Anaerobic |
| Fusobacterium | nucleatum | PD | Anaerobic |
| Treponema 2 | denticola, | PD | Anaerobic |
| | lecithinolyticum, | | |
| | maltophilum, medium, | | |
| | parvum, | | |
| | socranskii subsp. | | |
| | socranskii, spp. | | |
| | COT: 087, 170, | | |
| | 199, 201, 297, | | |
| | 233, 246, 247, 249, | | |
| | 325, 350, 352, | | |
| | 355, 356, 357, | | |
| | 358, 359, 397, | | |
| | FOT: 123, 144 | | |
| Staphylococcus | | Pathobiont | Facultatively |
| | | | Anaerobic |
| Clostridioides | difficile | Pathobiont | Anaerobic |
| Escherichia | coli | Pathobiont | Facultatively |
| | | | Anaerobic |
| Campylobacter | concisus, mucosalis, | Health/ | Anaerobic |
| | rectus | Pathobiont | |
| Corynebacterium | durum, mustelae, | Health | Aerobic |
| | COT 422, FOT | | |
| | 322 | | |
| Leucobacter | sp COT 429 | Health | Aerobic |
| Capnocytophaga | canimorsus, cynodegmi, | Health | Facultatively |
| | gingivalis, granulosa, | | Anaerobic |
| | ochracea, | | |
| | spp. | | |
| Bergeyella | | Health | Aerobic |
| Streptococcus | anginosus subsp. | Health | Facultatively |
| | anginosus, | | Anaerobic |
| | massiliensis 4401825, | | |
| | minor, | | |
| | mutans, ovis | | |
| | DSM16829, | | |
| | salivarius subsp. | | |
| | thermophilus, | | |
| | spp (COT: 279, 297, | | |
| | FOT: 345) | | |
| Fusibacter | | Health | Anaerobic |
| Conchiformibius | kuhniae, sp. COT289, | Health | Aerobic |
| Neisseria | steedae | Health | Aerobic |
| | canis, shayeganii, | | |
| | zoodegmatis, | | |
| | animaloris, weaveri | | |
| Frederiksenia | canicola, uncultured | Health | Facultatively |
| | bacterium | | Anaerobic |
| Pasteurella | dagmatis, multocida | Health | Aerobic |
| Moraxella | bovoculi, spp. | Health | Aerobic |

### Example 7: Using Categories to Assess Oral Health in Dogs and Cats

Oral health can be assessed based on microbiome community composition and the diversity and abundance of disease associated groups of anaerobic bacteria compared to aerobic health associates and the presence of potential pathogens/pathobionts. For each of nine dogs and eight cats, bacterial community composition was assessed by sequencing amplified DNA from the V4 region of the 16S rRNA gene on the Illumina MiniSeq platform, and measures determined as shown below in Table 3 (dogs) and Table 4 (cats).

Alpha diversity metrics (Amplified Sequence Variant Richness, Shannon Diversity, and Pielou's Evenness) were calculated for a subset of periodontal disease-associated bacteria (PD) and a subset of health-associated bacteria (HA). The range for PD richness in healthy cats and dogs is 0-39. The range for health richness in healthy cats and dogs is 20-50. For comparison, oral health of each individual was also assessed by examination by a veterinary technician and or a veterinarian and observations of oral health and breath were record as shown in the table below.

For each dog and each cat, for each metric, the ratio of the measure for PD to the measure for HA was calculated. Ratios as determined for the nine dogs were examined in relationship to the observed oral health status. For the richness and diversity measures for dogs, a threshold was identified that distinguished the ratios of dogs having good oral health from those having impaired oral health, including halitosis. For both richness and diversity, the threshold for dogs was 1.2. Similarly, for the richness and diversity measures for cats, a threshold was identified that distinguished the ratios of cats having good oral health from those having impaired oral health, including halitosis. For both richness and diversity, the threshold for cats was 1.2.

Thus, for both dogs and cats, individuals having a PD/HA richness and/or diversity measure above 1.2 had impaired oral health, and unfavorable microbiomes. Individuals having a PD/HA richness and/or diversity measure below 1.2 had good oral health and favorable microbiomes.

Pathobiont richness was determined in the same way as for HA and PD microbes. For this example, pathobiont richness was not associated with oral health. However, pathobiont richness could indicate colonization resistance of the oral microbiome, with lower values of richness indicating great resistance.

TABLE 3

| Metric | Dog 1 | Dog 2 | Dog 3 | Dog 4 | Dog 5 | Dog 6 | Dog 7 | Dog 8 | Dog 9 |
|---|---|---|---|---|---|---|---|---|---|
| Periodontal | 46 | 40 | 35 | 39 | 36 | 45 | 42 | 41 | 38 |
| Disease | | | | | | | | | |
| Associated | | | | | | | | | |
| (PD) Richness | | | | | | | | | |
| PD Diversity | 2.75 | 2.55 | 2.26 | 2.65 | 2.56 | 2.51 | 2.74 | 2.52 | 2.58 |
| PD Evenness | 0.72 | 0.69 | 0.63 | 0.72 | 0.71 | 0.76 | 0.75 | 0.68 | 0.74 |
| Health | 31 | 42 | 31 | 38 | 38 | 33 | 36 | 42 | 35 |
| Associated | | | | | | | | | |
| (HA) Richness | | | | | | | | | |
| Health | 2.06 | 2.32 | 2.65 | 2.57 | 2.84 | 1.78 | 2.35 | 2.65 | 2.34 |
| Diversity | | | | | | | | | |
| Health | 0.6 | 0.62 | 0.77 | 0.71 | 0.78 | 0.53 | 0.74 | 0.71 | 0.65 |
| Evenness | | | | | | | | | |
| Ratio: PD/HA | 1.48 | 0.95 | 1.13 | 1.03 | 0.95 | 1.36 | 1.17 | 0.98 | 1.09 |
| Richness | | | | | | | | | |
| PD/HA | 1.33 | 1.10 | 0.85 | 1.03 | 0.90 | 1.41 | 1.17 | 0.95 | 1.10 |
| Diversity | | | | | | | | | |
| PD/HA | 1.20 | 1.11 | 0.82 | 1.01 | 0.91 | 1.43 | 1.01 | 0.96 | 1.14 |
| Evenness | | | | | | | | | |
| Pathobiont | 1 | 1 | 2 | 2 | 2 | 3 | 2 | 1 | 1 |
| Richness | | | | | | | | | |
| Oral Health | Bleeding, | Healthy | Some | Healthy | Healthy | Adv. PD | Healthy | Healthy | Tartar |
| Observation | tartar, | gums, | tartar, | gums | gums | | gums, | gums | |
| | pockets | some | gums | | | | tartar | | |
| | | tartar | healthy | | | | | | |
| Breath | Halitosis | | Halitosis | | | Halitosis | Halitosis | | Halitosis |
| Assessment | | | | | | | | | |

TABLE 4

| Metric | Cat 1 | Cat 2 | Cat 3 | Cat 4 | Cat 5 | Cat 6 | Cat 7 | Cat 8 |
|---|---|---|---|---|---|---|---|---|
| PD Richness | 41 | 29 | 32 | 48 | 35 | 38 | 37 | 56 |
| PD Diversity | 2.55 | 2.51 | 2.76 | 2.49 | 2.41 | 2.94 | 2.54 | 3.29 |
| PD Evenness | 0.69 | 0.75 | 0.8 | 0.64 | 0.68 | 0.81 | 0.7 | 0.87 |
| HA Richness | 25 | 26 | 29 | 42 | 26 | 38 | 32 | 34 |
| HA Diversity | 1.77 | 2 | 2.24 | 2.47 | 2.14 | 2.52 | 2.68 | 2.51 |
| HA Evenness | 0.55 | 0.62 | 0.66 | 0.66 | 0.66 | 0.69 | 0.77 | 0.71 |
| PD/H Richness | 1.64 | 1.12 | 1.10 | 1.14 | 1.35 | 1.00 | 0.95 | 1.65 |
| PD/HA Diversity | 1.44 | 1.26 | 1.23 | 1.01 | 1.13 | 1.17 | 0.95 | 1.31 |
| PD/HA Evenness | 1.25 | 1.21 | 1.21 | 0.97 | 1.03 | 1.17 | 0.91 | 1.23 |
| Pathobiont | 2 | 3 | 1 | 3 | 2 | 2 | 1 | 4 |
| Richness | | | | | | | | |
| Oral Health | Bleeding | Some | Some | Healthy | Gum | Healthy | Healthy | Tartar |
| Observation | Tartar | tartar | tartar | gums | inflam- | gums | gums | Tooth |
| | Pockets | | | | mation | | | reabsorp- |
| | | | | | Bleeding | | | tion |
| Breath | Halitosis | | Halitosis | | | Halitosis | Halitosis | |
| Assessment | | | | | | | | |

### Example 8. Using Relative Abundance of Taxa, in View of Categories and Ranges

An example of comparing individual samples from domestic dogs and cats to a healthy reference database. Samples were collected by swabbing the area between the gingival sulcus and the gingival margin in cats and dogs in North America (USA and Canada). DNA was extracted using the QIAgen DNeasy PowerSoil Pro kit and samples prepared for sequencing on the Illumina MiniSeq and MiSeq platforms (Pichler et al. 2018). Raw sequence reads were filtered for quality control and processed using Qiime2 (Estaki et al. 2020) to generate an OTU-like table for bacterial genera and amplicon sequence variants. Taxonomic assignment was done using sklearn as part of Qiime2 against the Silva v132 database.

Results are shown below in Table 5 (dogs) and Table 6 (cats). Samples exceeding healthy reference ranges are highlighted in bold. For microbes associated with periodontal disease and/or pathobionts, overrepresentation may indicate an oral health impairment and the need for an intervention. For cats, additional genera that tended to be more common in cats than in dogs are included.

TABLE 5

| | | Range in | | | | | | | | |
|---|---|---|---|---|---|---|---|---|---|---|
| | | Healthy | | | | | | | | |
| Genus | Category | Dogs | Dog 1 | Dog 2 | Dog 3 | Dog 4 | Dog 5 | Dog 6 | Dog 7 | Dog 8 |
| Actinomyces | PD | 0-2 | 0 | 0.04 | 6.08 | 0.88 | 0.76 | 0.45 | 1.86 | 0.84 |
| Porphyromonas | PD | 0-3 | 4.73 | 4.94 | 6.35 | 5.34 | 2.71 | 3 | 1.46 | 3 |
| Parvimonas | PD | <1 | 1.22 | 1.45 | 0 | 0.29 | 0.34 | 0.03 | 0.45 | 1.41 |
| Peptostreptococcus | PD | <1 | 1.85 | 0.09 | 0.44 | 0.03 | 0 | 0.11 | 0.32 | 0.58 |
| Fusobacterium | PD | 0-6 | 6.64 | 7.86 | 2.14 | 4.65 | 4.26 | 1.22 | 2.65 | 4.19 |
| Treponema 2 | PD | 0-5 | 1.51 | 0.34 | 2.77 | 5.7 | 3.01 | 2.53 | 6.45 | 2.03 |
| Staphylococcus | PD | 0-4.5 | 0.3 | 1.86 | 0 | 0 | 0 | 0.01 | 0 | 0.02 |
| Clostridioides | Pathobiont | 0 | 0 | 0 | 0 | 0 | 0 | 0 | 2.15 | 0 |
| Escherichia | Pathobiont | 0-1 | 0.04 | 2.11 | 0 | 0 | 0 | 0 | 0 | 0 |
| Campylobacter | Pathobiont | 0-3 | 0.13 | 0.04 | 0.24 | 1.49 | 0.87 | 0.56 | 2.75 | 0.92 |
| Corynebacterium | Health | 0-5 | 0.16 | 0.37 | 0.28 | 0.67 | 0.14 | 0.31 | 0.26 | 1.11 |
| Leucobacter | Health | <1 | 0 | 0 | 0.07 | 0.06 | 0.01 | 0.55 | 0.8 | 0.15 |
| Capnocytophaga | Health | 0-9.5 | 0.58 | 1.41 | 2.19 | 1.34 | 0.91 | 9.43 | 0.5 | 2.72 |
| Bergeyella | Health | 0-10 | 0.33 | 1.44 | 1.13 | 1.19 | 3.97 | 9.98 | 1 | 3.83 |
| Streptococcus | Health | <1 | 0.08 | 0.22 | 0.77 | 0 | 0 | 1.24 | 0.12 | 0.49 |
| Fusibacter | Health | 0-4.5 | 2.28 | 4.47 | 0.14 | 1.91 | 0.38 | 0.98 | 2.02 | 1.17 |
| Conchiformibius | Health | 0-5 | 0 | 0.37 | 4.46 | 0.45 | 0.36 | 2.33 | 0.11 | 1.02 |
| Neisseria | Health | 0-6 | 0.22 | 0.27 | 0.71 | 1.66 | 4 | 2.13 | 0.35 | 1.91 |
| Frederiksenia | Health | 0-15 | 0.03 | 0.04 | 5.63 | 0.37 | 0.29 | 10.94 | 1.8 | 0.81 |
| Pasteurella | Health | 0-10 | 0.04 | 0.04 | 1.49 | 3.74 | 9.72 | 1.69 | 0.99 | 9.38 |
| Moraxella | Health | 0-20 | 4.77 | 9.18 | 21.16 | 11.73 | 5.6 | 18.98 | 3.49 | 14.08 |

TABLE 6

| | | Range in | | | | | | | | |
|---|---|---|---|---|---|---|---|---|---|---|
| | | Healthy | | | | | | | | |
| Genus | Category | Cats | Cat 1 | Cat 2 | Cat 3 | Cat 4 | Cat 5 | Cat 6 | Cat 7 | Cat 8 |
| Actinomyces | PD | 0-3 | 6.2 | 0.68 | 0.85 | 0.45 | 1.03 | 1.59 | 1.0 | 2.71 |
| Aggregatibacter | PD | <1 | 0 | 0 | 0 | 0.13 | 0.3 | 0 | 0 | 0 |
| Alloprevotella | PD | 0-5.5 | 0.91 | 4.5 | 0.68 | 0.54 | 0.96 | 0.26 | 0.91 | 3.61 |
| Bacteroides | PD | 0-5 | 6.72 | 4.10 | 3.08 | 2.47 | 1.47 | 0.95 | 5.72 | 3.08 |
| Prevotella | PD | 0-2 | 0 | 2.98 | 1.03 | 2.16 | 1.57 | 0.79 | 0.48 | 2.16 |
| Porphyromonas | PD | 0-15 | 11.0 | 23.0 | 14.2 | 5.05 | 18.1 | 11.4 | 22.9 | 14.2 |
| Parvimonas | PD | <1 | 0 | 0.01 | 0.05 | 0.01 | 0 | 0.02 | 0.05 | 0 |
| Peptostreptococcus | PD | 0-1 | 0.47 | 0 | 0 | 0 | 0.48 | 0 | 0 | 0 |
| Fusobacterium | PD | 0-2 | 0.21 | 2.36 | 1.73 | 0.56 | 2.27 | 4.07 | 3.62 | 2.21 |
| Treponema 2 | PD | 0-7 | 0.26 | 9.81 | 8.16 | 9.19 | 11.5 | 4.17 | 14.5 | 0.82 |
| Staphylococcus | PD | <1 | 0 | 0 | 0 | 0 | 0 | 0 | 0 | 0 |
| Clostridioides | Pathobiont | 0 | 0 | 0 | 0 | 0 | 0 | 0 | 0 | 0 |
| Escherichia | Pathobiont | 0 | 0 | 0 | 0 | 0.01 | 0 | 0 | 0 | 0 |
| Campylobacter | Pathobiont | <1 | 0.67 | 0.51 | 0.87 | 0.26 | 0.69 | 0.77 | 1.37 | 1.51 |
| Corynebacterium | Health | <1 | 0.60 | 0.05 | 0.11 | 0.10 | 0.18 | 0.42 | 1.20 | 0.45 |
| Leucobacter | Health | 0-3 | 4.72 | 0.08 | 0.20 | 0.16 | 0 | 0.51 | 0.07 | 0.18 |
| Capnocytophaga | Health | 0-3 | 4.82 | 1.52 | 1.46 | 1.72 | 0.75 | 4.41 | 1.20 | 1.78 |
| Bergeyella | Health | 0-2 | 0 | 4.35 | 2.65 | 15.1 | 0.31 | 7.26 | 0.21 | 0.47 |
| Streptococcus | Health | <1 | 0 | 0.03 | 0.06 | 0.16 | 0.06 | 0.05 | 0 | 0.87 |
| Fusibacter | Health | 0-2 | 0.19 | 1.54 | 3.07 | 0.45 | 2.17 | 0.75 | 2.86 | 1.33 |
| Conchiformibius | Health | 0-10 | 0 | 9.9 | 14.8 | 1.04 | 0.99 | 0.36 | 0 | 5.02 |
| Neisseria | Health | 0-2 | 0.10 | 0.33 | 0.46 | 0.61 | 1.28 | 1.55 | 1.45 | 1.32 |
| Frederiksenia | Health | 0-12 | 0.88 | 4.73 | 1.35 | 15.03 | 0.36 | 3.24 | 1.10 | 2.09 |
| Pasteurella | Health | 0-14 | 23.9 | 3.26 | 4.74 | 2.10 | 9.56 | 10.13 | 1.28 | 5.09 |
| Moraxella | Health | 0-17 | 0 | 7.4 | 18.9 | 11.5 | 12.7 | 17.4 | 8.32 | 0.03 |

### Example 9: Cats

Microbial diversity metrics were assessed for three sequential samples collected from a cat with advanced periodontal disease with tooth resorption prior to a dental cleaning to remove calculus, tooth extraction, and treatment with cefovecin sodium, a broad-spectrum antibiotic. Microbial diversity measures for samples collected from a cat with advanced periodontal disease with tooth resorption prior to a dental procedure that included cleaning to remove calculus, tooth extraction, and antibiotic treatment. Samples were collected by swabbing the area between the gingival sulcus and the gingival margin and were sequenced using the Illumina MiniSeq. As shown in Table 7 below, the dental treatment reduced richness and diversity of both PD and HA microbes, and the PD/HA ratios for richness and diversity increased from before the procedure to two weeks after. At five weeks after, PD richness had increased but PD diversity had decreased, likely driven by a reduction in evenness. At five weeks after, for HA microbes, there were increases in richness, diversity, and evenness, and the PD/HA ratios for richness and diversity had decreased. Thus, this cat's oral microbiome began to show signs of recovery in a sample collected five weeks later.

TABLE 7

| Microbial | Before | Two weeks | Five weeks |
|---|---|---|---|
| Diversity Metric | dental | after | after |
| PD Richness | 41 | 19 | 23 |
| PD Diversity | 2.26 | 2.17 | 1.86 |
| PD Evenness | 0.53 | 0.74 | 0.62 |
| HA Richness | 31 | 13 | 25 |
| HA Diversity | 1.87 | 1.11 | 1.62 |
| HA Evenness | 0.62 | 0.43 | 0.58 |
| PD/HA Richness | 1.32 | 1.46 | 0.92 |
| PD/HA Diversity | 1.21 | 1.95 | 1.15 |
| PD/HA Evenness | 0.85 | 1.72 | 1.07 |
| Pathobiont | 3 | 2 | 2 |
| Richness | | | |

### Example 10: Before Vs after for Dogs

The efficacy of oral health interventions is assessed using sequential sampling. Samples of the oral microbiome of several individual dogs were obtained prior to and after a course of prebiotic supplements. Prebiotic powdered supplements were added to drinking water on a daily basis between the times of sampling. Bacteria in the microbiome samples were divided into the following categories: Favorable: aerobic and or oral health-associated (HA) and Unfavorable: anaerobic, periodontal disease-associated (PD), and or potential pathogen (pathobiont). Relative abundance was determined for each of various taxa in each category.

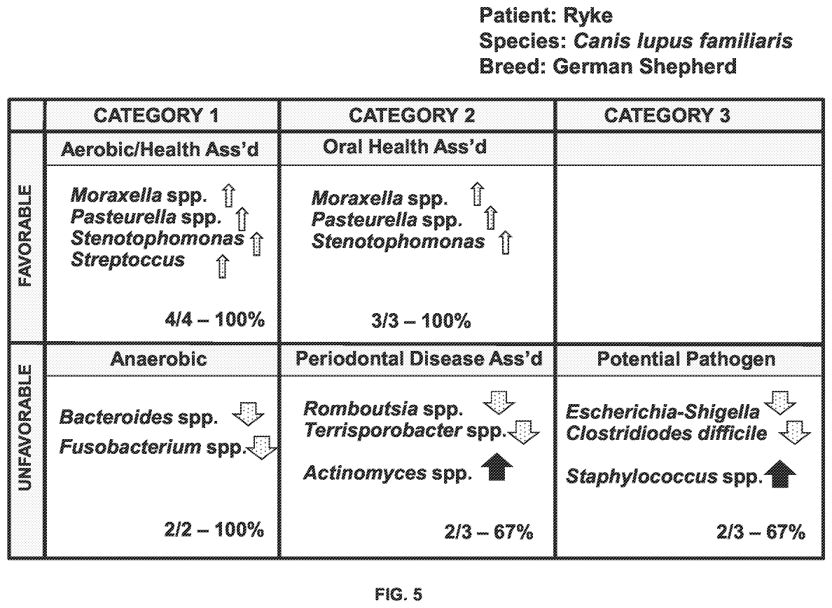

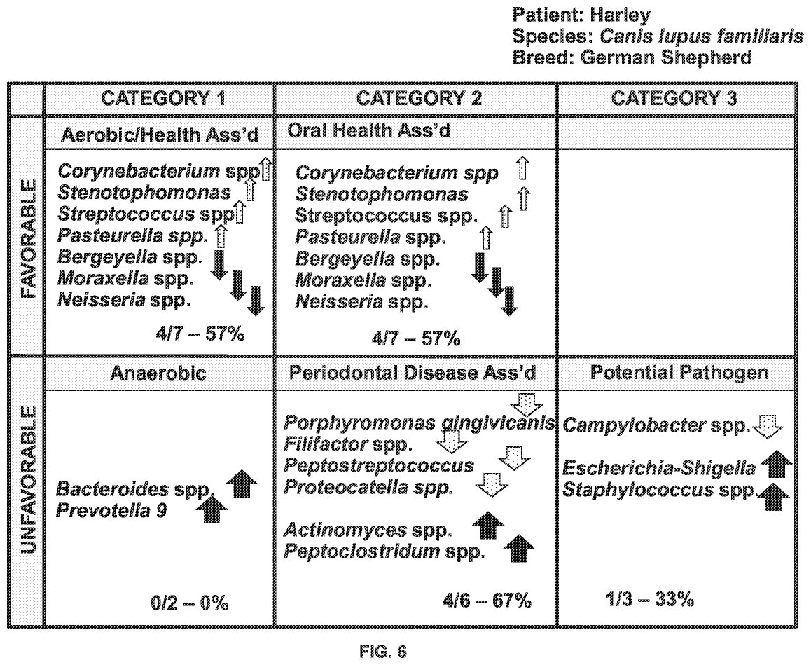

Results are shown in FIG. 5 for a first dog, and in FIG. 6 for a second dog.

As shown in FIG. 5, for the first dog, the community shifted towards aerobic respiration: four groups of favorable aerobic bacteria increased in relative abundance and two groups of unfavorable anaerobic bacteria decreased out of a total of 6 groups present in the samples. The community showed increased representation of HA bacteria and a reduction in PD bacteria: four out of 4 HA bacteria increased and two out of three PD bacteria decreased. Three potential pathogens (pathobionts) were detected and two decreased and another increased. This suggests that the oral biofilm may be providing improved resistance to colonization by pathobionts. Overall, the community shifted towards greater representation of favorable groups of bacteria.

As shown in FIG. 6, for the second dog, the community response to the oral health intervention was mixed. Out of seven groups of favorable bacteria detected, four increased while three groups decreased in relative abundance. While four groups of PD bacteria decreased following the intervention, two groups of anaerobic bacteria increased and an additional two PD bacteria increased in relative abundance. Three potential pathogens were detected and two increased. Overall, these results suggest that an escalation in oral care may be warranted or that additional time may be needed to assess the efficacy of the intervention.

### In-Home Testing Kit

Embodiments of the invention encompass products for administration to affect such microbes, including compositions for oral administration.

An apparatus for in-home testing of the response of the oral biofilm to interventions to improve oral health is also provided. The testing kit includes swabs used to collect the biofilm at the gumline for genetic characterization using PCR and or sequencing, as well as characterization of volatile metabolites using chemical analyses, such as gas chromatography-mass spectrometry. Genomic sequencing is used to characterize the composition of the biofilm and to predict metabolic processes of the oral microbiome biofilm community.

In some embodiments, the oral microbiome kit includes a product to improve oral health, such as toothpaste or a prebiotic supplement to manipulate the composition of bacteria in the oral microbiome.

### Definitions

The phrase “as used herein” encompasses all of the specification, the abstract, the drawings (figures), and the claims.

As used herein, the use of the singular includes the plural and vice versa unless expressly stated to be otherwise, or as obvious from the context that such is not intended. That is, “a,” “an” and “the” refer to one or more of whatever the word modifies. For example, “a sample” may refer to one sample, two samples, etc. Likewise, “the sample” may refer to one, two or more samples. By the same token, words such as, without limitation, “samples” would refer to one sample as well as to a plurality of samples unless it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. With the preceding discussion in mind, a numerical value herein that is modified by a word of approximation may vary from the stated value by ±15% in some embodiments, by ±10% in some embodiments, by ±5% in some embodiments, or in some embodiments, may be within the 95% confidence interval. As used herein, all numbers which represent physical values or measurements are subject to the standard error in the measurement of the value.

As used herein, any ranges presented are inclusive of the end-points. For example, “an abundance between 10% and 30%” or “an abundance from 10% to 30%” includes 10% and 30%, as well as any percentages in between. In addition, throughout this disclosure, various aspects of this invention may be presented in a range format. The description in range format is merely for convenience and brevity and should not be construed as an inflexible limitation on the scope of the invention. Accordingly, the description of a range should be considered to have specifically disclosed all the possible subranges as well as individual numerical values within that range. As an example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. Unless expressly indicated, or from the context clearly limited to integers, a description of a range such as from 1 to 6 should be considered to have specifically disclosed subranges 1.5 to 5.5, etc., and individual values such as 3.25, etc. that is non-integer individual values and ranges beginning with, ending with or both beginning with and ending with non-integer value(s). This applies regardless of the breadth of the range.

As used herein, the word “about” may be used to characterize a particular value. When values are expressed as approximations by use of the antecedent “about,” it will be understood that the particular value forms another embodiment. As a non-limiting example, if “from about 1 to about 4” is disclosed, another embodiment is “from 1 to 4,” even if not expressly disclosed. Likewise, if one embodiment disclosed is a temperature of “about 30%,” then another embodiment is “30%,” even if not expressly disclosed. Similarly, numbers or ranges presented as a specific value or specific range also encompass another embodiment in which the number or the end of the range is preceded by “about.” As a non-limiting example, if “an abundance of 30%” is expressly disclosed, then another embodiment is “an abundance of about 30%,” even if not expressly disclosed. In a similar manner, if “from 1 to 4” is disclosed, another embodiment is “from about 1 to about 4,” even if not expressly disclosed.

As used herein, the use of “preferred,” “preferably,” or “more preferred,” and the like to describe an embodiment refers to preferences as they existed at the time of filing of the patent application.

As used herein, the phrase “combination of” preceded by “a” or “any” and followed by a list joined by the conjunction “and” (and obvious variants of these), means any combination of two or more members of the group where the group members are the members of the list joined by the conjunction “and.” As a non-limiting example, “any combination of A, B, C, and D” encompasses the following combinations: A and B; A and C; A and D; B and C; B and D; C and D; A, B, and C; A, B, and D; A, C, and D; B, C, and D; A, B, C, and D. Similarly, for a list ending with or including “combinations thereof” (or obvious variants such as “all combinations thereof”), the above definition also applies. As a non-limiting example, the phrase “X is selected from the group consisting of A, B, C, D, and combinations thereof” means X is A, X is B, X is C, X is D, or X is “any combination of A, B, C, and D” where the above definition of “any combination thereof” applies. Likewise, a phrase such as “X is A, B, C, D, or a combination thereof” means X is A, X is B, X is C, X is D, or X is “a combination of A, B, C, and D” where the above definition of “a combination thereof” applies.

As used herein, the phrase “and/or” means a combination or an individual member. As a non-limiting example, “X is A, B, and/or C” encompasses the following possibilities: X is A; X is B; X is C; X is any combination of A, B, and C (A and B; A and C; B and C; A, B, and C).

As used herein, “treatment of an oral disease and/or oral condition” includes, but is not limited to including, administration of a dietary component to and/or physical intervention with an individual having the oral disease and/or oral condition in a manner sufficient to have a beneficial effect on the oral health of the individual, including (but not limited to) curing, slowing progression of, causing retrogression of, and alleviating one or more symptoms of the oral disease or oral condition.

As used herein, “prophylactic treatment” includes, but is not limited to including, administration of a dietary component to and/or physical intervention with an animal at risk of having the oral disease and/or oral condition in a manner sufficient to have a beneficial effect on the oral health of the individual, including (but not limited to): (1) preventing or delaying on-set of the oral disease and/or oral condition in the first place; (2) maintaining an oral disease and/or oral condition at a retrogressed level; and (3) preventing or delaying recurrence of oral disease and/or oral condition after a course of treatment.

### REFERENCES CITED

Banerjee, S., Siburt, C. J. P., Mistry, S., Noto, J. M., DeArmond, P., Fitzgerald, M. C., Lambert, L. A., Cornelissen, C. N. and Crumbliss, A. L. 2012. Evidence of Fe 3+ interaction with the plug domain of the outer membrane transferrin receptor protein of Neisseria gonorrhoeae: implications for Fe transport. Metallomics 4(4): 361-372.
Darveau, R. P., Hajishengallis, G., Curtis, M. A. 2012. Porphyromonas gingivalis as a potential community activist for disease. J Dent Res. 91: 816-820.
Davis, I. J., Wallis, C., Deusch, O., Colyer, A., Milella, L., Loman, N., Harris, S. 2013. A cross-sectional survey of bacterial species in plaque from client owned dogs with healthy gingiva, gingivitis or mild periodontitis. PloS One 8(12):e83158.
Dhar, V. and Tinanoff, N. 2017. Update on Sugar Alcohols and Their Role in Caries Prevention. Here's what the evidence says about the potential oral health benefits of commonly used sugar alcohols. Last updated Feb. 8, 2017; available at https://decisionsindentistry.com/article/update-sugar-alcohols-role-caries-prevention/
Eley, B. M. and Cox, S. W., 2003. Proteolytic and hydrolytic enzymes from putative periodontal pathogens: characterization, molecular genetics, effects on host defenses and tissues and detection in gingival crevice fluid. Periodontology 31(1): 105-124.
Fournier, D., Mouton, C., Lapierre, P., Kato, T., Okuda, K., Ménard, C. 2001. Porphyromonas gulae sp. nov., an anaerobic, gram-negative coccobacillus from the gingival sulcus of various animal hosts. International Journal of Systematic and Evolutionary Microbiology 51(3): 1179-89.
Holcombe, L. J., Patel, N., Colyer, A., Deusch, O., O'Flynn, C. and Harris, S., 2014. Early canine plaque biofilms characterization of key bacterial interactions involved in initial colonization of enamel. PLoS One, 9(12), p.e113744.
Kačírová, J., Mad'ar, M., Štrkolcová, G., Mad'ari, A. and Nemcová, R., 2019. Dental Biofilm as Etiological Agent of Canine Periodontal Disease. In Bacterial Biofilms, IntechOpen.
Khurshid, Z., Mali, M., Naseem, M., Najeeb, S. and Zafar, M. S., 2017. Human gingival crevicular fluids (GCF) proteomics: an overview. Dentistry Journal 5(1): 12.
O'Flynn, C., Deusch, O., Darling, A. E., Eisen, J. A., Wallis, C., Davis, I. J. and Harris, S. J., 2015. Comparative genomics of the genus Porphyromonas identifies adaptations for heme synthesis within the prevalent canine oral species Porphyromonas cangingivalis. Genome Biology and Evolution 7(12): 3397-3413.
Olczak, T., Simpson, W., Liu, X. and Genco, C. A., 2005. Iron and heme utilization in Porphyromonas gingivalis. FEMS Microbiology Reviews 29(1): 119-144.
Ratledge, C. and Dover, L. G. 2000. Iron metabolism in pathogenic bacteria. Annual Reviews in Microbiology 54(1): 881-941.
Würsch, P. and Koellreutter, B., 1982. Maltitol and maltotriitol as inhibitors of acid production in human dental plaque. Caries Research 16(2): 90-95.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method for modifying an oral microbiome of a companion mammal, the method comprising:
obtaining a test sample from an oral tissue or oral cavity of the companion mammal, the test sample including microbes of the oral microbiome;
analyzing the test sample to obtain test data for a plurality of taxa of the microbes of the oral microbiome of the companion mammal;
determining at least one test measure for each of one or more microbiome features based on the test data;
obtaining at least one reference measure for each of the one or more microbiome features, the at least one reference measure based on analysis of one or more reference samples from oral tissues or oral cavities of one or more reference companion mammals, the one or more reference samples including microbes of one or more reference oral microbiomes, the analysis of the one or more reference samples providing reference data for a plurality of taxa of microbes, the at least one reference measure based on the reference data;
based on the at least one test measure and the at least one reference measure for the one or more microbiome features, identifying a first intervention to modify the oral microbiome of the companion mammal.

2. The method of claim 1, wherein the at least one reference measure is based on analysis of one or more reference samples from the oral tissue or oral cavity of the companion mammal.

3. The method of claim 1, wherein the at least one reference measure is based on analysis of one or more reference samples from oral tissues or oral cavities of each of two or more reference companion mammals.

4. The method of claim 1 or 3, wherein the reference companion mammals are companion mammals characterized by oral health or periodontal disease state.

5. The method of any of claims 1-4, wherein the test sample is obtained after a preliminary intervention.

6. The method of claim 5, wherein at least one of the one or more reference samples is obtained before the preliminary intervention.

7. The method of claim 5 or 6, wherein at least one of the one or more reference samples is obtained after the preliminary intervention.

8. The method of any of claim 5 or 6, wherein the one or more reference samples are obtained after the preliminary intervention.

9. The method of any of claims 5-8, wherein the first intervention is the same as the preliminary intervention.

10. The method of any of claims 5-8, wherein the first intervention is different from the preliminary intervention.

11. The method of any of claims 5-8, wherein the first intervention is in addition to the preliminary intervention.

12. The method of any of claims 1-11 and 15-37, wherein the one or more microbiome features comprise an indicator of taxonomic richness or diversity for each of two or more categories of microbes.

13. The method of claim 12, wherein the one or more microbiome features comprise a ratio of taxonomic richness or diversity for periodontal disease-associated microbes to taxonomic richness or diversity for oral health-associated microbes.

14. The method of any of claims 1-11 and 15-37, wherein the reference data provides a threshold value for assessing the at least one test measure for each of one or more microbiome features.

15. The method of any of claims 1-14, wherein the first intervention is selected from administration of a probiotic, prebiotic fiber, prebiotic supplement, bacteriophage, bacteriophage-associated protein, bacteriophage lysins, antimicrobial, antimicrobial peptide, nanoparticles with biocidal properties, sodium bicarbonate, chelating agents, glycoproteins, phototherapy, and any combination thereof.

16. The method of any of claims 1-14, wherein the first intervention comprises administration of a chelating agent and prebiotics.

17. The method of any of claims 1-16, wherein the first intervention comprises modification of a dietary component.

18. The method of any of claims 1-17, wherein the first intervention is selected from tooth brushing, tooth flossing, mechanical removal or reduction in biofilm, dental cleaning, tooth extraction, root canal, dental examination, administration of an antibiotic, and any combinations thereof.

19. The method of any of claims 5-11, wherein the preliminary intervention is selected from administration of a probiotic, prebiotic fiber, prebiotic supplement, bacteriophage, bacteriophage-associated protein, bacteriophage lysins, antimicrobial, antibiotic, antimicrobial peptide, nanoparticles with biocidal properties, sodium bicarbonate, chelating agents, glycoproteins, phototherapy, and any combination thereof.

20. The method of any of claims 5-11, wherein the preliminary intervention comprises administration of a chelating agent and prebiotics.

21. The method of any of claims 5-11 and 19-20, wherein the preliminary intervention comprises modification of a dietary component.

22. The method of any of claims 5-11 and 19-21, wherein the preliminary intervention is selected from tooth brushing, tooth flossing, mechanical removal or reduction in biofilm, dental cleaning, tooth extraction, root canal, dental examination and any combinations thereof.

23. The method of claim 5, wherein identifying the first intervention includes assessing the success of the preliminary intervention.

24. The method of claim 23, wherein success of the preliminary intervention is indicated by one or more of an increase in aerobic bacteria, an increase in oral-health associated bacteria, a decrease in anaerobic bacteria and a decrease in periodontal disease-associated bacteria.

25. The method of claim 23, wherein success of the preliminary intervention is indicated by a decrease in pathogen/pathobiont bacteria.

26. The method of claim 23, wherein assessing the success of the preliminary intervention includes assessing whether there is an improvement in a measure of microbes in at least one category.

27. The method of claim 26, wherein the at least one category is selected from aerobic bacteria, anaerobic bacteria, periodontal-associated bacteria, pathogen/pathobiont bacteria, and oral-health associated bacteria.

28. The method of claim 23, wherein assessing the success of the preliminary intervention includes determining one or more of an increase in taxonomic diversity, taxonomic richness, and taxonomic evenness.

29. The method of any of claims 23-28, wherein assessing the success of the preliminary intervention includes consideration of more or more of the companion mammal's diet, general health, exposure to microbes, dental care history, and oral hygiene.

30. The method of claim 29, wherein oral hygiene comprises one or more of health of teeth, health of gums, health of tongue, low or no plaque and tartar, periodontal health, and fresh breath.

31. The method of any of claims 1-30, further comprising identifying a second intervention.

32. The method of claim 31, wherein the second intervention is selected from administration of a probiotic, prebiotic fiber, prebiotic supplement, bacteriophage, bacteriophage-associated protein, bacteriophage lysins, antimicrobial, narrow-spectrum antibiotic, antimicrobial peptide, nanoparticles with biocidal properties, sodium bicarbonate, chelating agents, glycoproteins, phototherapy, and any combination thereof.

33. The method of claim 30, wherein the second intervention is selected from tooth brushing, tooth flossing, mechanical removal or reduction in biofilm, dental cleaning, tooth extraction, root canal and dental examination.

34. The method of claim 30, wherein the second intervention is the same as the first intervention.

35. The method of claim 30, wherein the first intervention is determined to be unsuccessful.

36. The method of claim 34, wherein the second intervention is different than the first intervention.

37. The method of any of claims 1-35, wherein the at least one test measure and the at least one reference measure each comprise a value, a set of values, a range of values, a normalized value, a parameter or a set of parameters for a statistic, metric, or model, or any combination thereof.

38. The method of any of claims 1-37, wherein the at least one test measure and the at least one reference measure each comprise one or more indicators of taxonomic diversity, taxonomic richness, and taxonomic evenness.

39. The method of any of claims 1-37, wherein the one or more microbiome features comprises an indication of similarity and/or relatedness of two or more of the plurality of taxa of the microbes of the oral microbiome.

40. The method of any of claims 1-37, wherein the one or more microbiome features reflect one or more aspects of the composition of microbes in the oral microbiome or changes in the composition of microbes in the oral microbiome.

41. The method of any of claims 1-37, wherein the one or more microbiome features comprise one or more measures of relative abundance of one or more of the plurality of taxa of the microbes of the oral microbiome of the companion mammal.

42. The method of any of claims 1-37, wherein the at least one test measure and the at least one reference measure for each of the one or more microbiome features indicates presence of two or more of the plurality of taxa, or presence or absence of two or more of a plurality of predefined taxa.

43. The method of any of claims 1-37, wherein the one or more microbiome features reflect one or more aspects of the function of microbes in the oral microbiome or changes in the microbial metabolic products associated with a microbiome.

44. The method of claim 43, wherein the one or more microbiome features indicate activity for one or more metabolic categories of microbes in the oral microbiome.

45. The method of claim 44, wherein the one or more microbiome features comprise an indicator of anaerobic bacteria and an indicator of aerobic bacteria.

46. The method of any of claims 1-37, wherein the one or more microbiome features comprise an indicator of periodontal disease-associated bacteria or an indicator of pathogen/pathobiont bacteria.

47. The method of any of claims 1-37, wherein the one or more microbiome features comprise an indicator of periodontal disease-associated bacteria and an indicator of oral health-associated bacteria.

48. The method of any of claims 1-37, wherein the one or more microbiome features comprise indicators for two or more of anaerobic bacteria, aerobic bacteria, periodontal disease-associated bacteria, oral-health-associated bacteria, and pathogen/pathobiont bacteria.

49. The method of claim prior 1-37, wherein the one or more microbiome features comprise indicators for three or more of anaerobic bacteria, aerobic bacteria, periodontal disease-associated bacteria, oral-health-associated bacteria, and pathogen/pathobiont bacteria.

50. The method of any of claims 1-37, wherein the one or more microbiome features further comprises a measure of two or more taxa of microbes.

51. The method of any of claims 1-37, wherein each of the one or more microbiome features comprises and a presence, an amount, or a relative abundance of microbes according to taxa or category.

52. The method of any of claims 1-37, wherein each of the one or more microbiome features indicate a change in presence, amount, or relative abundance of microbes according to taxa or category.

53. The method of any of claims 1-37, wherein each of the one or more microbiome features indicates the relative abundance or a change in relative abundance of aerobic bacteria, anaerobic bacteria, periodontal disease-associated bacteria and pathogen/pathobiont bacteria.

54. The method of any of claims 1-53, wherein the analyzing comprises analyzing genetic material in the test sample and each of the one or more reference samples.

55. The method of claim 54, wherein the analyzing further comprises identifying the plurality of taxa of microbes by sequence analysis.

56. The method of claim 55, wherein identifying the plurality of taxa of microbes is accomplished by shotgun sequencing of extracted deoxyribonucleic acid (DNA) and/or by targeted sequencing of microbial diversity.

57. The method of claim 54, wherein the genetic material includes ribonucleic acid (RNA) from microbes of the oral microbiome of the companion mammal.

58. The method of claim 54, wherein the genetic material includes deoxyribonucleic acid (DNA) from microbes of the oral microbiome of the companion mammal.

59. The method of any of claims 1-53, wherein the analyzing comprises analyzing proteins in the test sample and each of the one or more reference samples.

60. The method of any of claims 1-59, wherein the analyzing further comprises characterization of volatile metabolites using chemical analysis.

61. The method of any of claims 54-60, wherein the analyzing further comprises characterizing the composition or metabolic function of the oral microbiome for a biofilm of the companion mammal.

62. The method of any of claims 1-61, further comprising providing a plan for the companion mammal's oral health.

63. The method of any of claims 1-61, further comprising implementing the first intervention and modifying the oral microbiome of the companion mammal.

64. The method of claim 63, wherein modifying the oral microbiome of the companion mammal comprises improving at least one of the one or more microbiome feature of the oral microbiome of the companion mammal.

65. The method of any of claims 1-61, further comprising providing materials for executing the first intervention.

66. The method of any of claims 1-61, wherein the first intervention modifies the oral microbiome of the companion mammal by improving the balance of microbes of the oral microbiome, by increasing favorable microbes, and/or by decreasing unfavorable microbes.

67. The method of any of claims 1-61, further comprising preserving the test sample.

68. The method of claim 67, wherein preserving the test sample includes one or more of drying the test sample, placing the test sample in a buffer, and combining the test sample with a preservative.

69. A method of assessing an oral microbiome sample from a companion mammal comprising:
obtaining a test sample from an oral tissue or cavity of the companion mammal, the test sample including microbes of the oral microbiome;
preparing an assay sample comprising nucleic acid from the test sample;
sequencing a portion of the nucleic acid to identify microbes in the test sample according to two or more taxa;
identifying the presence and/or level of microbes for each of the two or more taxa;
assessing the test sample based upon the presence and/or level of microbes for each of the two or more taxa according to one or more categories of microbes.

70. The method of claim 68, wherein the one or more categories are selected from anaerobic, aerobic, periodontal disease-associated, oral health associated, and potential pathogen/pathobiont.

71. The method of claim 69, further comprising identifying a plan for the companion's oral health based on the assessment.

72. The method of any of claims 1-71, wherein the companion mammal is a dog.

73. The method of any of claims 1-71, wherein the companion mammal is a cat.

74. The method of any of claims 1-71, wherein the companion mammal and the one or more reference companion mammals are of a same species.

75. The method of claim 74, wherein the companion mammal and at least one of the one or more reference companion mammals are of a same breed.

76. The method of claim 75, wherein the companion mammal and at least one of the one or more reference companion mammals are of different breeds.

77. The method of any of claims 1-76, further comprising providing materials for obtaining the test sample.
